(19) United States
(12) Patent Application Publication
(10) Pub. No.: US 2026/0102251 A1
(43) Pub. Date: Apr. 16, 2026

(54) TWO COMPONENT INTRODUCER ASSEMBLY

(71) Applicant: Edwards Lifesciences Corporation, Irvine, CA (US)

(72) Inventor: Tamir S. Levi, Zikhron Yaakov (IL)

(73) Assignee: Edwards Lifesciences Corporation, Irvine, CA (US)

(21) Appl. No.: 19/422,268

(22) Filed: Dec. 16, 2025

Related U.S. Application Data
(63) Continuation of application No. PCT/US2024/037010, filed on Jul. 8, 2024.
(60) Provisional application No. 63/526,715, filed on Jul. 14, 2023.

(51) Int. Cl. A61F 2/24

(52) U.S. Cl. CPC A61F 2/2436

(57) ABSTRACT

The present disclosure is direct to an introducer assembly for expanding a sheath. The introducer assembly including an introducer and a core member received within the central lumen of the introducer. The introducer is movable between a first and second position in response to the outwardly directed radial force exerted on the central lumen of the introducer by an expansion element provided on the core member.

### CROSS-REFERENCE TO RELATED APPLICATIONS

This application is a continuation of International Application No. PCT/2024/037010, filed July 8, 2024, and entitled “Two Component Introducer Assembly,” which claims the benefit of U.S. Provisional Application No. 63/526,715, filed July 14, 2023, the contents of which are incorporated herein by reference in their entirety.

### FIELD

The present application is directed to an expandable sheath and introducer for use with catheter-based technologies for repairing and/or replacing heart valves, as well as for delivering an implant, such as a prosthetic valve to a heart via the patient’s vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient’s vasculature (for example, the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device.

One method to reduce push forces required to advance the delivery device through the sheath is to pre-dilate the sheath and/or strain relief portion by passing a relatively large dilator (for example, 22 French dilator) into the sheath. This is done during sheath prep, prior to sheath insertion into the patient and/or with the sheath at least partially inserted into the patient. The challenge with this method is that it can be difficult with regard to physical strength of the user (for example, grip and arm strength) to advance the dilator into sheath. Additionally, it is important that the dilator pass all the way to the distal end sheath while also avoiding splitting of the sheath and/or distal end of the sheath, which could cause difficulty or vessel injury during the delivery device insertion/removal process.

Accordingly, there remains a need for devices, systems, and methods of providing a sheath including a strain relief portion, that allows the sheath body and distal tip to expand reducing the initial push force when introducing the delivery system and implant.

### SUMMARY

Aspects of the present expandable sheath and introducer system can minimize trauma to the vessel and damage to the sheath and prosthetic device by reducing push forces through the sheath. Some aspects ensure that the sheath is not damaged during efforts to dilate or expand the strain relief portion. Additional aspects of the present expandable sheath can minimize trauma to the vessel by allowing for temporary expansion of a portion of the introducer sheath to accommodate a delivery system, followed by a return to the original diameter once the delivery system passes through. Some aspects can comprise a sheath with a smaller profile than that of prior art introducer sheaths. Furthermore, certain implementations can reduce the length of time a procedure takes, as well as reduce the risk of a longitudinal or radial vessel tear, or plaque dislodgement, because lower push force is required and only one sheath is used, rather than several different sizes of sheaths.

An implementation of the present disclosure provides an introducer assembly including an elongated introducer including a central lumen and an elongated core member received within the central lumen of the introducer and movable between a first and second position. The central lumen of the introducer extends between a proximal opening at proximal end of the introducer and a distal opening at a distal end of the introducer. The core member includes: an elongated body portion, and an expansion element extending radially from an outer surface of the body portion. At least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the central lumen of the introducer by the expansion element as it moved between the first and second positions.

In some implementations, when the core member is in the first position, at least a portion of the expansion element and/or the body portion extends through the distal opening, and when the core member is in the second position, the expansion element is positioned within the central lumen of the introducer such that contact between the expansion element and the central lumen provides an outwardly directed radial force on the central lumen moving the introducer from the unexpanded configuration to the expanded configuration.

In some implementations, the core member includes a central lumen extending between a proximal end and distal end of the core member, for example, for receiving a guide wire.

In some implementations, the expansion element includes distal tapered surface. In in some implementations, the distal tapered surface is adjacent the distal end of the of the core member to facilitate advancement of the introducer assembly, in the unexpanded configuration, through the patient’s blood vessel.

In some implementations, the expansion element includes a proximal tapered surface extending between the elongated body portion of the core member and an increased diameter portion of the expansion element. In some implementations, the proximal tapered surface facilitates sliding movement between the introducer and the expansion element as the core member is moved proximally during introducer expansion.

In some implementations, the expansion element has a barrel-shaped longitudinal cross section.

In some implementations, the introducer includes a plurality of slits extending longitudinally along the introducer and extending radially from an inner surface of the central lumen, where separation/widening of at least one of the plurality of slits facilitates expansion of the introducer from the unexpanded configuration to the expanded configuration, and where plurality of slits are evenly spaced around a circumference of the central lumen. In some implementations, the plurality of slits extend along a portion of the length of the introducer. In further implementations, the plurality of slits extend along the entirety of the length of the introducer.

In some implementations, the introducer assembly further includes an introducer locking mechanism for fixing the axial/longitudinal location of the introducer with respect to the core member, and a sheath locking mechanism for fixing the axial/longitudinal location of the introducer with respect to the sheath.

A further implementation of the present disclosure provides a method of expanding a sheath comprising: positioning an elongated core member within a central lumen of an elongated introducer in a first position, where the core member includes an elongated body portion and an expansion element extending radially from an outer surface of the body portion; advancing the core member and the introducer into a central lumen of an expandable sheath; expanding the introducer by moving the core member into a second position such that the introducer moves from an unexpanded configuration in which the introducer has a first outer diameter to an expanded configuration in which the introducer has a second, larger, outer diameter due to an outwardly directed radial force exerted on the central lumen of introducer by the expansion element as it moved from the first position to the second position; and expanding the expandable sheath by moving the core member within the central lumen of the introducer such that contact between the expansion element and the central lumen provides an outwardly directed radial force on the central lumen moving the introducer from the unexpanded configuration to the expanded configuration resulting in a corresponding radial expansion along a length of the sheath.

In some implementations, positioning the core member at the first position within the central lumen of the elongated introducer includes advancing the core member within the introducer such that at least a portion of the expansion element and/or the body portion extends through a distal opening of the introducer.

In some implementations, expanding the expandable sheath further includes moving the core member and introducer together (for example, proximally) within the central lumen of the sheath such that contact between the expanded introducer and the sheath results in a corresponding radial expansion of the sheath (for example, the sheath is expanded as the introducer assembly including the core member and introducer together are removed from the sheath).

In some implementations, the method further includes coupling the core member to the introducer in the first position. In some implementations, the core member is coupled to the introducer using the introducer locking mechanism before and/or after being advanced into the sheath, fixing the axial and/or rotational position of the core member with respect to the introducer and preventing corresponding movement therebetween.

In some implementations, before expanding the expandable sheath by moving the core member to the second position, the introducer is coupled to the sheath. In some implementations, the introducer is coupled to the proximal hub of the sheath via the sheath locking mechanism, fixing the axial and/or rotational position of the introducer with respect to the sheath and preventing corresponding movement therebetween.

In some implementations, the method further includes: advancing the combined core member, introducer and sheath into a patient’s blood vessel (for example, femoral artery) with the core member in the first position with respect to the introducer; expanding a length of the blood vessel by expanding a corresponding length of the expandable sheath; withdrawing the introducer and core member from the blood vessel; and advancing a medical device through the central lumen of the sheath and beyond a distal opening of the sheath to a treatment site, where the medical device is a prosthetic heart valve mounted in a radially crimped state on a delivery apparatus (for example, balloon catheter), and the method further comprises implanting the prosthetic heart valve at the treatment site within the patient.

Another implementation of the present disclosure provides an introducer assembly including an elongated introducer and an elongated core member. The elongated introducer including: a central lumen that extends between a proximal opening at proximal end of the introducer and a distal opening at a distal end of the introducer; and an inner shoulder extending radially inward from the central lumen. The elongated core member is received within the central lumen of the introducer and movable between a first and second position, the core member including an elongated body portion; where at least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the inner shoulder of the introducer by the core member as it moved between the first and second positions.

In some implementations, when the core member is in the first position, the distal end of the core member is positioned proximal of the inner shoulder, and when the core member is in the second position, the core member is positioned adjacent the inner shoulder such that contact between the core member and the inner shoulder provides an outwardly directed radial force on the introducer moving the introducer from the unexpanded configuration to the expanded configuration.

In some implementations, the core member includes a central lumen extending between a proximal end and distal end of the core member (for example, for receiving a guide wire), where the core member has a constant outer diameter along a length of the core member, and where the core member includes a distal tapered surface to facilitate advancement of the core member along the inner shoulder.

In some implementations, the inner shoulder extends longitudinally from a distal end of the introducer and defines a reduced diameter portion of the central lumen having a diameter less than a diameter of a main diameter portion of the central lumen when the introducer is in the unexpanded configuration.

In some implementations, the inner shoulder has a proximal tapered surface extending between the main diameter portion and the reduced diameter portion facilitating sliding movement between the core member and the inner shoulder as the core member is moved distally during introducer expansion.

In some implementations, the introducer includes a plurality of slits extending longitudinally along the inner shoulder and extending radially from an inner surface of the central lumen, where separation/widening of at least one of the plurality of slits facilitates expansion of the introducer from the unexpanded configuration to the expanded, where the plurality of slits are evenly spaced around a circumference of the central lumen.

In some implementations, the introducer assembly further includes an introducer locking mechanism for fixing the axial/longitudinal location of the introducer with respect to the core member, and a sheath locking mechanism for fixing the axial/longitudinal location of the introducer with respect to the sheath.

A further implementation of the present disclosure provides a method of expanding a sheath comprising: positioning an elongated core member within a central lumen of an elongated introducer in a first position, the introducer including: a central lumen that extends between a proximal opening at proximal end of the introducer, a distal opening at a distal end of the introducer; and an inner shoulder extending radially inward from the central lumen; coupling the core member to the introducer 210 in the first position (for example, fixing the position of the core member with respect to the introducer by engaging the introducer locking mechanism); advancing the core member and the introducer into a central lumen of an expandable sheath; coupling the introducer (and/or core member) to the expandable sheath (for example, fixing the position of the unexpanded introducer assembly with respect to the sheath by engaging the sheath locking mechanism); uncoupling the core member from the introducer such that the core member is capable of freely moving (for example, axially and rotationally) with respect to the introducer (for example, by disengaging the introducer locking mechanism); expanding the introducer by moving the core member from the first position, where a distal end of the core member is positioned proximal of the inner shoulder, into a second position, where the core member is advanced with the central lumen and in contact with the inner shoulder, such that the introducer moves from an unexpanded configuration in which the introducer has a first outer diameter to an expanded configuration in which the introducer has a second, larger, outer diameter due to an outwardly directed radial force exerted on the inner shoulder by the core member; uncoupling the introducer (and/or core member) from the expandable sheath (for example, by disengaging the sheath locking mechanism); and expanding the expandable sheath by moving the core member and the introducer in the expanded configuration (for example, withdrawing the expanded introducer assembly proximally) within the central lumen of the sheath such that contact between the expanded introducer and the sheath results in a corresponding radial expansion of the sheath (for example, the sheath is expanded as the introducer assembly including the core member and introducer together are removed from the sheath).

In some implementations, the method further includes: advancing the combined core member, introducer and sheath into a patient’s blood vessel (for example, femoral artery) with the core member in the first position with respect to the introducer; expanding a length of the blood vessel by expanding a corresponding length of the expandable sheath; withdrawing the introducer and core member from the blood vessel; and advancing a medical device through the central lumen of the sheath and beyond a distal opening of the sheath to a treatment site, where the medical device is a prosthetic heart valve mounted in a radially crimped state on a delivery apparatus (for example, balloon catheter), and the method further comprises implanting the prosthetic heart valve at the treatment site within the patient.

According to some implementations, a sheath for introducing a prosthetic device wherein the sheath comprises an inner liner and an outer layer is disclosed. At least a portion of the sheath can be designed or configured to locally expand from a first diameter (rest diameter) to a second diameter (expanded diameter) as the prosthetic device is pushed through the lumen of the sheath, and then at least partially return to the first diameter once the prosthetic device has passed through.

Various aspects of the implementations described above can be combined based on desired sheath system characteristics.

### BRIEF DESCRIPTION OF DRAWINGS

FIG. 1 illustrates a delivery system for a cardiovascular prosthetic device, according to one implementation.

FIG. 2 illustrates an expandable sheath that can be used in combination with the delivery system of FIG. 1, according to one implementation.

FIG. 3 is a side elevation cross-sectional view of an introducer assembly and the expandable sheath of FIG. 2, in an unexpanded configuration.

FIG. 4 is a side elevation cross-sectional view of the introducer assembly and expandable sheath of FIG. 3 in an expanded configuration.

FIG. 5 is a side elevation cross-sectional view of an introducer assembly and the expandable sheath of FIG. 2, in an unexpanded configuration.

FIG. 6 is a side elevation cross-sectional view of the introducer assembly and expandable sheath of FIG. 5 in an expanded configuration.

FIG. 7 is a partial side elevation cross-sectional view of the introducer of FIG. 5.

FIG. 8 is a magnified view of a portion of the expandable sheath of FIG. 2.

FIG. 9 is a side elevation cross-sectional view of a portion of the expandable sheath of FIG. 2.

FIG. 10A is a magnified view of a portion of the expandable sheath of FIG. 2, with the outer layer removed for illustration purposes.

FIG. 10B is a magnified view of a portion of the braided layer of the sheath of FIG. 2.

FIG. 11 is a magnified view of a portion of the expandable sheath of FIG. 2, illustrating the expansion of the sheath as a prosthetic device is advanced through the sheath.

FIGS. 12A-12D illustrate implementations of a braided layer in which the filaments of the braided layer are configured to buckle when the sheath is in a radially collapsed state.

FIG. 13 is a cross-section of an expandable sheath implementation.

FIG. 14 is a simplified cross-section of a sheath wall having a plurality of longitudinally extending pleats in one implementation.

FIGS. 15A-15B are photographs of the sheath in a collapsed state (FIG. 15A) and an expanded state (FIG. 15B).

FIG. 16 is a simplified cross-section of a distal portion of a sheath wall having a plurality of longitudinally extending pleats in one implementation.

FIG. 17 is a simplified cross-section of a distal portion of a sheath wall having a plurality of longitudinally extending pleats in one implementation.

FIG. 18 is a cross-section of an expandable sheath in a proximal portion of the sheath in one implementation.

FIG. 19 is a schematic of the sheath of FIG. 18.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

The present disclosure relates to introducer sheaths. Such introducer sheaths may be radially expandable. However, currently known sheaths tend to have complex mechanisms, such as ratcheting mechanisms that maintain the sheath in an expanded configuration once a device with a larger diameter than the sheath’s original diameter is introduced. Existing expandable sheaths can also be prone to axial elongation as a consequence of the application of longitudinal force attendant to passing a prosthetic device through the sheath. Such elongation can cause a corresponding reduction in the diameter of the sheath, increasing the force required to insert the prosthetic device through the narrowed sheath.

Accordingly, there remains a need in the art for an improved introducer sheath for endovascular systems used for implanting valves and other prosthetic devices.

### Definitions

As used in this application and the claims, the singular forms “a,” “an,” and “the” include the plural forms unless the context clearly dictates otherwise. Thus, for example, reference to a “polymer” includes aspects having two or more such polymers unless the context clearly indicates otherwise.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable combination.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and the claims, the term “comprising” can include the aspects “consisting of” and “consisting essentially of.” Additionally, the term “includes” means “comprises.”

For the terms “for example” and “such as,” and grammatical equivalences thereof, the phrase “and without limitation” is understood to follow unless explicitly stated otherwise.

References in the specification and concluding claims to parts by weight of a particular element or component in a composition or article denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5 and are present in such ratio regardless of whether additional components are contained in the composition.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, including the recited values, may be used. Further, ranges can be expressed herein as from “about” one particular value and/or to “about” another particular value. When such a range is expressed, some aspects include from one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (for example, within 2% or 1%) of the particular value modified by the term “about.”

As used herein, the term "and/or" includes any and all combinations of one or more of the associated listed items. It is also understood that the term “and/or” includes where one or another of the associated listed items is present, and the aspects where both of the associated listed items are present, or any combinations of the associated listed items are present.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “substantially” means that the subsequently described event or circumstance completely occurs or that the subsequently described event or circumstance generally, typically, or approximately occurs.

As used herein, the term “substantially,” when used in reference to a composition or a compound, refers to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” in, for example, the context “substantially free” refers to a composition having less than about 1 % by weight, for example, less than about 0.5 % by weight, less than about 0.1 % by weight, less than about 0.05 % by weight, or less than about 0.01 % by weight of the stated material, based on the total weight of the composition.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component.

Further, the terms “coupled” and “associated” generally mean electrically, electromagnetically, and/or physically (for example, mechanically or chemically) coupled or linked and do not exclude the presence of intermediate elements between the coupled or associated items.

It will be understood that, although the terms “first,” "second," etc., may be used herein to describe various elements, components, regions, layers sections, and/or steps. These elements, components, regions, layers, sections, and/or steps should not be limited by these terms. These terms are only used to distinguish one element, component, region, layer, or section from another element, component, region, layer, section, or step. Thus, a first element, component, region, layer, section, or step discussed below could be termed a second element, component, region, layer, section, or step without departing from the teachings of example aspects.

It is understood that the terms “layer” and “liner” can be used interchangeably. For example, the aspects describing an “inner liner” also include aspects describing an “inner layer.” Similarly, the aspects describing an “outer layer” also include aspects describing an “outer liner.”

Spatially relative terms, such as "beneath," "below," "lower," "above," "upper," and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as "below" or "beneath" other elements or features would then be oriented "above" the other elements or features. Thus, the term "below" can encompass both an orientation of above and below. The device may be otherwise oriented (rotated 90 degrees or at other orientations), and the spatially relative descriptors used herein are interpreted accordingly.

As used herein, the term “atraumatic” is commonly known in the art and refers to a device or a procedure that reduces tissue injury.

Some of the implementations disclosed herein comprise a plurality of longitudinally-extending creases. It is understood that the terms “creases,” “folds,” and “pleats” as used in reference to these implementations can be used interchangeably. It is understood that the pleats or creases can be arranged in a specific pattern, or they can be randomly formed along a length of the sheath. For example, pleats formed along the length of the sheath are formed as a result of the manufacturing process where the various polymer layers encapsulate a braid (or braided layer; it is understood that braid and braided layer can be used interchangeably) and form creases that can be flattened out during expansion process of the heart valve. In some implementations, pleats can have an arranged pattern. For example, and without limitation, pleats can have an arranged pattern at the tip of the sheath. In some implementations, the pleats can have an even area in an arc length of a cross section. In such implementations, each of the formed pleats can have a substantially identical area, whether there are 2 pleats, or 3 pleats, or 4 pleats, or 5 pleats, and so on. In some implementations, the pleats can have a predetermined design for the desired application.

It is understood that the term “collapsed” as used herein refers to a natural unexpanded state of the sheath.

Although the operations of exemplary implementations of the disclosed method may be described in a particular sequential order for convenient presentation, it should be understood that disclosed implementations can encompass an order of operations other than the particular sequential order disclosed. For example, operations described sequentially may, in some cases, be rearranged or performed concurrently. Further, descriptions and disclosures provided in association with one particular implementation are not limited to that implementation and may be applied to any implementation disclosed.

While implementations of the present disclosure can be described and claimed in a particular statutory class, such as the system statutory class, this is for convenience only, and one of ordinary skill in the art will understand that each implementation of the present disclosure can be described and claimed in any statutory class. Unless otherwise expressly stated, it is in no way intended that any method or implementation set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of implementations described in the specification.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present disclosure may be understood more readily by reference to the following detailed description of various implementations of the disclosure and the examples included therein and to the Figures and their previous and following description.

Disclosed herein are elongate introducer sheaths that are particularly suitable for delivery of implants in the form of implantable heart valves, such as balloon-expandable implantable heart valves. Expandable introducer sheaths are disclosed in U.S. Patent No. 8,690,936, entitled “Expandable Sheath for Introducing an Endovascular Delivery Device into a Body,” U.S. Patent No. 8,790,387, entitled “Expandable Sheath for Introducing an Endovascular Delivery Device into a Body,” U.S. Patent No. 10,639,152, entitled “Expandable Sheath and Methods of Using the Same,” U.S. Patent No. 10,792,471, entitled “Expandable Sheath,” U.S. Patent No. Application No. 16/407,057, entitled “Expandable Sheath with Elastomeric Cross Sectional Portions,” U.S. Patent No. 10,327,896, entitled “Expandable Sheath with Elastomeric Cross Sectional Portions,” U.S. Patent No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. Application No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Patent No. 11,051,939, entitled “Active Introducer Sheath System,” Application No. PCT/US2022/012684, entitled “Introducer with Sheath Tip Expander,” U.S. Application No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

Balloon-expandable implantable heart valves are well-known and will not be described in detail here. An example of such an implantable heart valve is described in U.S. Patent No. 5,411,552, and also in U.S. Patent No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject’s vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything – prosthetic or not – that is delivered to a site within a body. A diagnostic device, for example, may be an implantable.

Disclosed implementations of an expandable sheath can minimize trauma to the vessel by allowing for temporary expansion of a portion of the introducer sheath to accommodate the delivery system, followed by a return to the original diameter once the device passes through. The expandable introducer sheaths described herein can be used to deliver a prosthetic device through a patient's vasculature to a procedure site within the body. The sheath can be constructed to be highly expandable and collapsible in the radial direction while limiting axial elongation of the sheath and, thereby, undesirable narrowing of the lumen. In one implementation, the expandable sheath includes a braided layer, one or more relatively thin, non-elastic polymeric layers, and an elastic layer. The sheath can resiliently expand from its natural diameter to an expanded diameter as a prosthetic device is advanced through the sheath and can return to its natural diameter upon passage of the prosthetic device under the influence of the elastic layer. In certain implementations, the one or more polymeric layers can engage the braided layer and can be configured to allow radial expansion of the braided layer while preventing axial elongation of the braided layer, which would otherwise result in elongation and narrowing of the sheath.

As will be described in more detail herein, the sheath is pre-dilated (for example, dilated in advance of the delivery system insertion through the sheath) using the two-component introducer assembly described herein. The implementations described herein avoid the need for multiple insertions for the dilation of the vessel by using a two-component introducer assembly that functions as both an introducer and a dilator. Present implementations reduce the length of time a procedure takes, as well as reduce the risk of a longitudinal or radial vessel tear, or plaque dislodgement because only one sheath is required, rather than several different sizes of sheaths, introducers, and/or dilators. Moreover, the pull forces required to facilitate movement of the introducer assembly and expansion of the sheath by the expanded end of the introducer assembly can be significantly smaller than the push forces required to push a conventional dilator with a uniform diameter along its length.

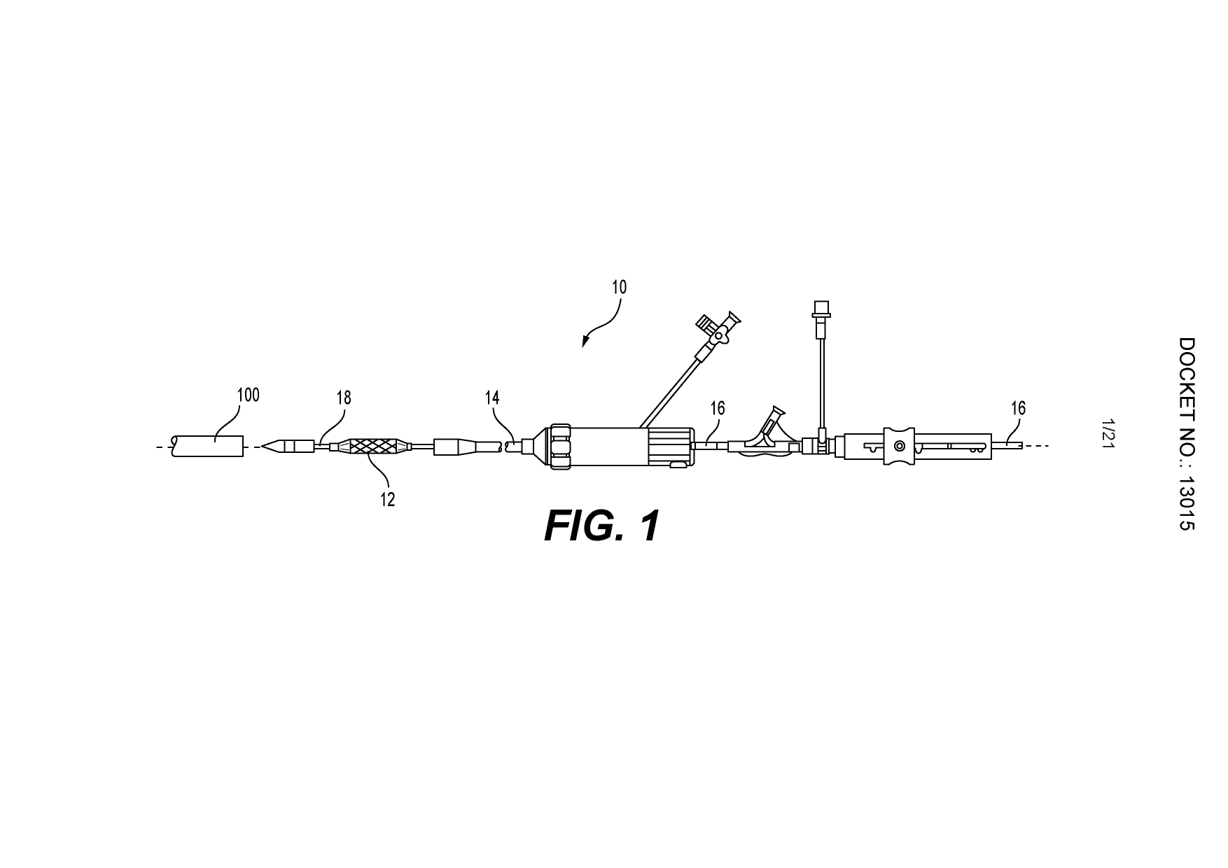

FIG. 1 illustrates a representative delivery apparatus 10 for delivering a medical device 12, such as a prosthetic heart valve or other prosthetic implant, to a patient. The delivery apparatus 10 is exemplary only and can be used in combination with any of the expandable sheath implementations described herein. Likewise, the sheaths disclosed herein can be used in combination with any of the various known delivery apparatuses. The delivery apparatus 10 illustrated can generally include a steerable guide catheter 14 and a balloon catheter 16 extending through the guide catheter 14. A medical device 12, such as prosthetic device (for example, a prosthetic heart valve), can be positioned on the distal end of the balloon catheter 16. The guide catheter 14 and the balloon catheter 16 can be adapted to slide longitudinally relative to each other to facilitate the delivery and positioning of a prosthetic heart valve (medical device 12) at an implantation site in a patient's body. The guide catheter 14 includes a handle portion 18 and an elongated guide tube or shaft 20 extending from the handle portion 18.

The prosthetic heart valve (medical device 12) can be delivered into a patient’s body in a radially compressed configuration and radially expanded to a radially expanded configuration at the desired deployment site. In the illustrated implementation, the prosthetic heart valve is a plastically expandable prosthetic valve that is delivered into a patient’s body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Some details regarding a plastically expandable heart valve that can be implanted using the devices disclosed herein are disclosed in U.S. Publication No. 2012/0123529, which is incorporated herein by reference. In some implementations, the prosthetic heart valve can be a self-expandable heart valve that is restrained in a radially compressed configuration by a sheath or other component of the delivery apparatus 10 and self-expands to a radially expanded configuration when released by the sheath or other component of the delivery apparatus 10. Some details regarding a self-expandable heart valve that can be implanted using the devices disclosed herein are disclosed in U.S. Publication No. 2012/0239142, which is incorporated herein by reference. In some implementations, the prosthetic heart valve can be a mechanically expandable heart valve that comprises a plurality of struts connected by hinges or pivot joints and is expandable from a radially compressed configuration to a radially expanded configuration by actuating an expansion mechanism that applies an expansion force to the prosthetic heart valve.

Some details regarding a mechanically expandable heart valve that can be implanted using the devices disclosed herein are disclosed in U.S. Publication No. 2018/0153689, which is incorporated herein by reference. In some implementations, a prosthetic heart valve (medical device 12) can incorporate two or more of the above-described technologies. For example, a self-expandable heart valve can be used in combination with an expansion device to assist the expansion of the prosthetic heart valve.

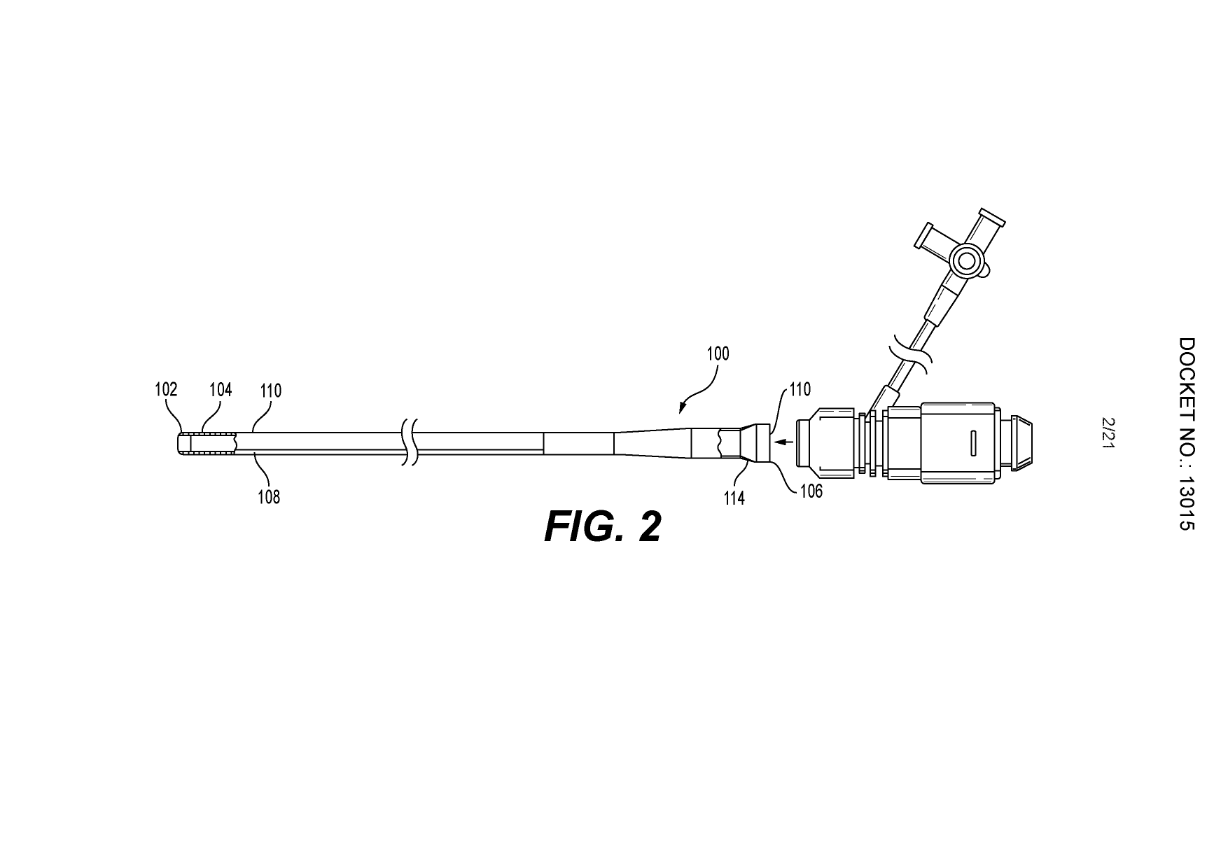

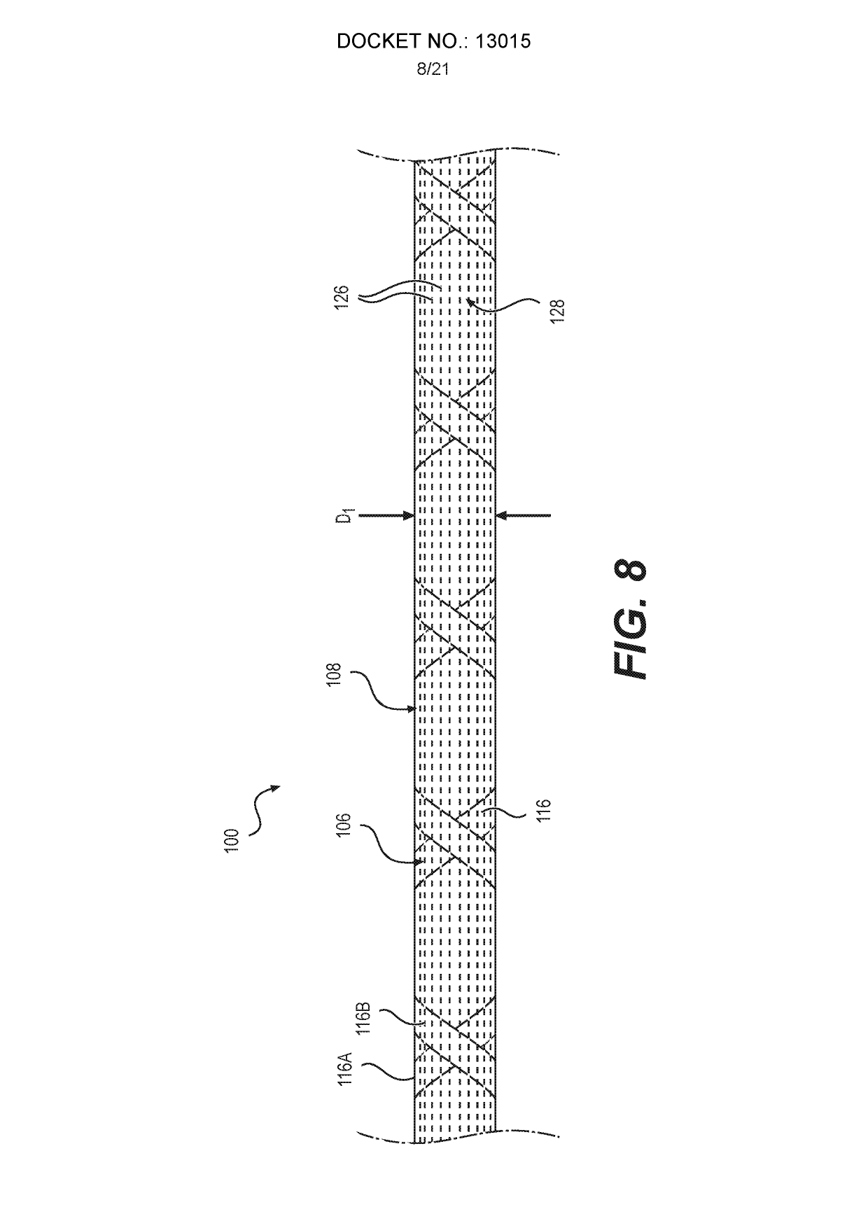

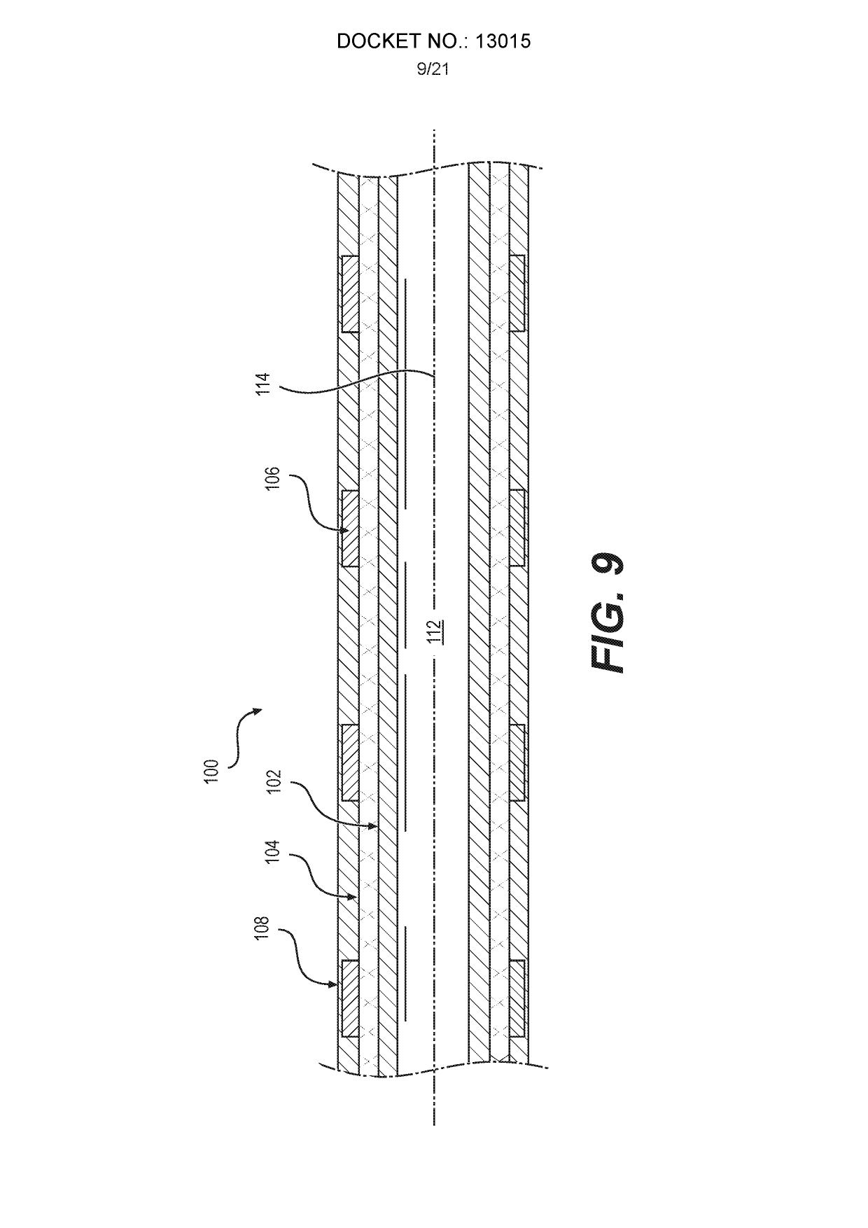

FIG. 2 illustrates an assembly 90 (which can be referred to as an introducer device or assembly) that can be used to introduce the delivery apparatus 10 and the prosthetic device (medical device 12) into a patient's body. The introducer device 90 can comprise a housing 92 at a proximal end of the introducer device 90 and an expandable sheath 100 extending distally from the housing 92. The housing 92 can function as a handle for the device. The expandable sheath 100 has a central lumen 112 (FIG. 9) to guide the passage of the delivery apparatus 10 for the prosthetic heart valve (medical device 12). An example implementation of the expandable sheath 100 is described in more detail herein in reference to FIGS. 8-19. In some implementations, the introducer device 90 need not include a housing 92. For example, the sheath 100 can be an integral part of a component of the delivery apparatus 10, such as the guide catheter. For example, the sheath 100 can extend from the handle portion 18 of the guide catheter. Additional examples of introducer devices and expandable sheaths can be found in U.S. Patent Application No. 16/378,417 and U.S. Provisional Patent Application No. 62/912,569, which are incorporated by reference in their entireties.

Generally, during use, a distal end of the sheath 100 is passed through the patient's skin and inserted into a vessel, such as the femoral artery. The delivery apparatus 10 with its medical device 12 (prosthetic heart valve 12) can then be inserted through the housing 92 and the sheath 100 and advanced through the patient's vasculature to the treatment site, where the medical device 12 is to be delivered and implanted within the patient. In certain implementations, the introducer housing 92 can include a hemostasis valve that forms a seal around the outer surface of the guide catheter 14 once inserted through the housing to prevent leakage of pressurized blood.

In some implementations, to reduce push force through the sheath 100, the sheath 100 and/or blood vessel is dilated, for example, pre-dilated, before the delivery apparatus 10 is advanced through the sheath 100. In some implementations, the sheath 100 is pre-dilated by passing a relatively large dilator (for example, 22 French dilator) through the sheath 100. This can be done during sheath 100 preparation, prior to sheath 100 insertion into the patient and/or with the sheath 100 at least partially inserted into the patient. However, this method requires significant physical strength of the user (for example, grip and arm strength) to advance the dilator through the sheath 100 and/or patient’s blood vessel. In some implementations, it may be desirable to pre-dilate the entire length of the sheath 100, including the distal tip portion. The distal tip of the sheath 100 resists radial expansion and requires increased push force to advance the delivery device beyond and/or through the distal opening of the sheath 100.

The two-component introducer assembly 200 described herein, avoids the need for multiple, different sized, introducers and/or dilators to be advanced through the sheath 100. The introducer assembly 200 allows the sheath 100 to be dilated after insertion into the patient and/or with the sheath 100 partially inserted into the patient. The introducer assembly 200 eliminates the need to dilate the sheath 100 during preparation, reduces preparation time, and eliminates unnecessary opportunities for error and/or inadvertent damage to the sheath 100 and the patient’s blood vessel.

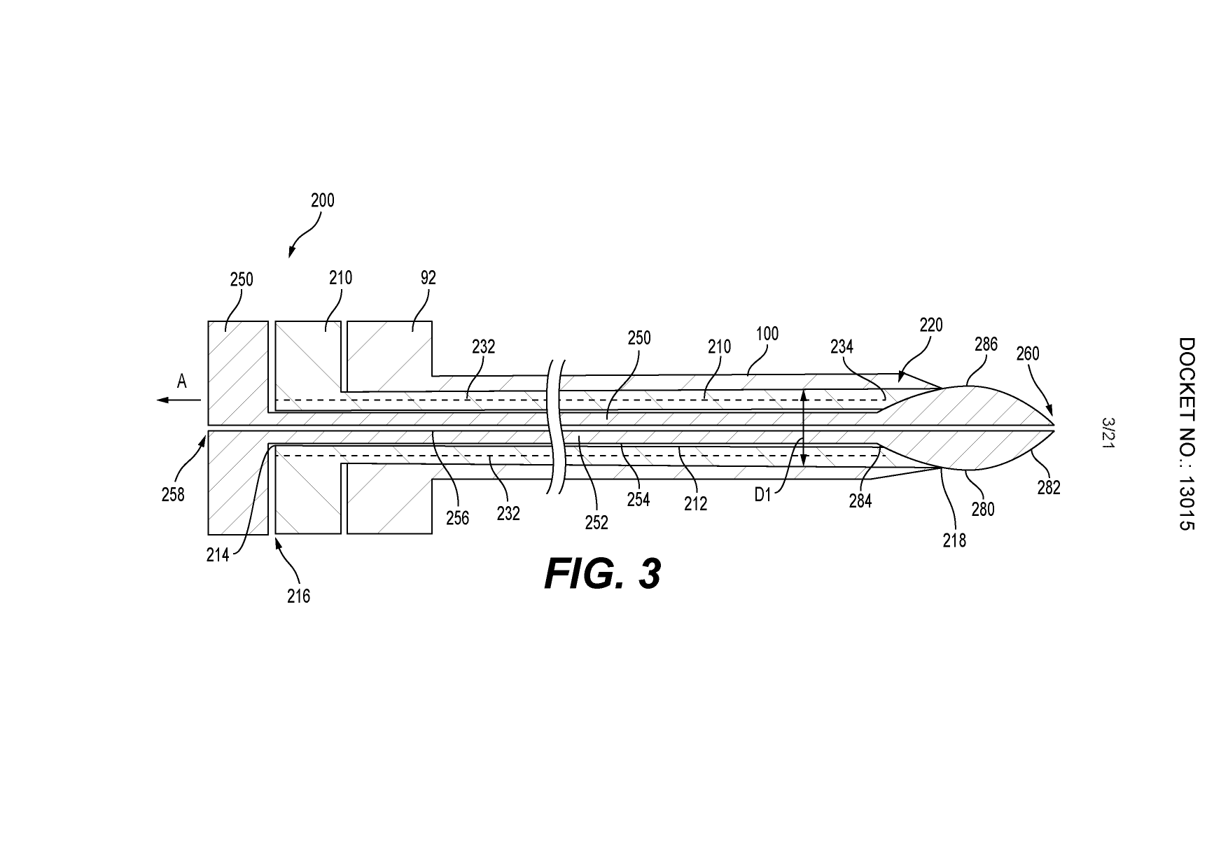

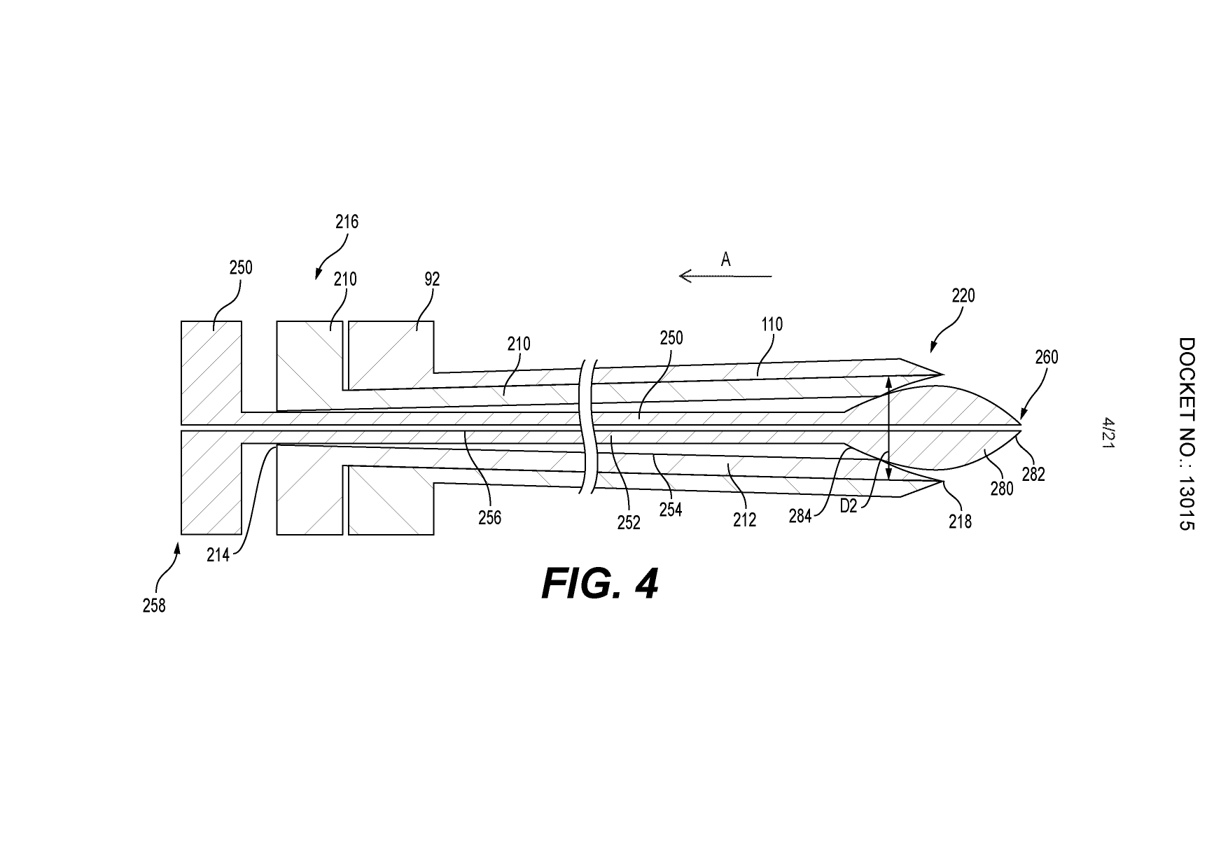

FIGS. 3 and 4 illustrate an example introducer assembly 200 configured to radially expand from an unexpanded configuration (FIG. 3) to an expanded configuration (FIG. 4). With the introducer assembly 200 in the expanded configuration, it is withdrawn from the central lumen of the sheath 100 and expands a corresponding portion/length the sheath 100. FIG. 3 is a side elevation cross-sectional view of the example introducer assembly 200 received within the expandable sheath 100 of FIG. 2. As shown in FIG. 3, the introducer assembly is in an unexpanded configuration. FIG. 4 is a side elevation cross-sectional view of the introducer assembly 200 received within the expandable sheath 100 in an expanded configuration.

The introducer assembly 200 includes an elongated introducer 210 and an elongated core member 250. The core member 250 is received within the central lumen 212 of the introducer 210 which extends between a proximal opening 214 at the proximal end 216 of the introducer 210 and a distal opening 218 at the distal end 220 of the introducer 210. The core member 250 is sized and configured to be movably received within the central lumen 212 of the introducer 210 and moveable between a first position (FIG. 3) and a second position (FIG. 4), resulting in the corresponding movement of the introducer 210 between an unexpanded and expanded configuration.

FIG. 3 illustrates the core member 250 received within the central lumen 212 of the introducer 210 in the first position where the introducer 210/introducer assembly 200 is in a corresponding unexpanded configuration. In the unexpanded configuration, the introducer 210 has a first outer diameter (D1). In some implementations, the first outer diameter (D1) is less than or equal the outer diameter of the unexpanded expandable sheath 100. FIG. 4 illustrates the core member 250 received within the central lumen 212 of the introducer 210 in the second position where the introducer 210 is in a corresponding expanded configuration. In the expanded configuration, the introducer 210 has a second larger outer diameter (D2), greater than the first outer diameter (D1). As the core member 250 moves in a proximal direction from the first position to the second position, the introducer 210 moves from the unexpanded to the expanded configuration in response to the outwardly directed radial force exerted on the central lumen 212 of the introducer 210 by the expansion element 280 provided on the core member 250.

As illustrated in FIGS. 3 and 4, the core member 250 includes an elongated body portion 252 and a central lumen 256 extending between the proximal end 258 and distal end 260 of the core member 250. In some implementations, the central lumen 256 is sized and configurated to receive a guide wire used to direct placement of the introducer assembly 200/sheath 100 within the patient’s blood vessel.

As provided in FIG. 3, when the core member 250 is in the first position, at least a portion of the expansion element 280 and/or the body portion 252 extends through the distal opening 218. Illustrated in FIG. 4, as the core member 250 is moved in a proximal direction (direction A) to the second position, the expansion element 280 moves within the central lumen 212 of the introducer 210 such that contact between the expansion element 280 and the central lumen 212 provides the outwardly directed radial force on the central lumen 212, thereby expanding the introducer 210 and moving the introducer 210 from the unexpanded configuration to the expanded configuration.

The expansion element 280 projects radially from the outer surface 254 of the body portion 252 of the core member 250. In other words, the expansion element has an outer diameter that is greater than the outer diameter of the body portion 252 of the core member 250. In some implementations, the expansion element 280 is defined as a regular or irregular shaped projection extending from the outer surface of the core member 250. In some implementations, the expansion element 280 can extend around all or a portion of the circumference of the core member 250. In some implementations, the expansion element 280 has a barrel-shaped longitudinal cross section. In other implementations, the expansion element 280 has an elliptical, egg-shaped, and/or circular-shaped longitudinal cross section. In some implementations, the outer diameter of the expansion element 280 ranges from 12Fr to 22Fr, or from 14Fr to 22Fr. In some implementations, the outer diameter of the expansion element 280 is approximately 22Fr, and the outer diameter of the introducer 210 in the expanded configuration is approximately 24 Fr.

As illustrated in FIGS. 3 and 4, in some implementations, the expansion element 280 includes distal tapered surface 282. The distal tapered surface 282 is located adjacent/proximate the distal end 260 of the core member 250. In some implementations, the distal tapered surface 282 extends from the distal end 260 of the of the core member 250 in a direction toward the proximal end 258 of the core member 250 to facilitate advancement of the introducer assembly 200, in the unexpanded configuration, through the patient’s blood vessel. In some implementations, the distal tapered surface 282 includes a curved outer surface, for example, a convex outer surface. In some implementations, as illustrated in FIG. 3, the expansion element 280 has smooth other surface. For example, the expansion element 280 provides a smooth surface extending longitudinally from the distal end 260 to the elongated body portion 252 of the core member 250. In some implementations, the expansion element 280 provides a smooth transition between proximal tapered surface 284 of the core member 250 and the elongated body portion 252 if the core member 250. Similarly, in some implementations, the expansion element 280 provides a smooth surface extending circumferentially round the expansion element 280/core member 250. The smooth outer surface of the core member 250 facilitates movement of the delivery apparatus 10, including the introducer assembly 200 and sheath 100, through the patient’s blood vessel.

As illustrated in FIGS. 3 and 4, in some implementations, the expansion element 280 includes a proximal tapered surface 284. The proximal tapered surface 284 extends from the elongated body portion 252 in a direction toward the distal end of the core member 250 to facilitate withdrawal of the core member 250 proximally within the central lumen 212 of the introducer 210, (for example, by easing the sliding movement between the introducer 210 and the expansion element 280 as the core member 250 is moved proximally during introducer 210 expansion). In some implementations, the proximal tapered surface 284 extends between the elongated body portion 252 of the core member 250 and the increased diameter portion 286 of the expansion element 280, the increased diameter portion 286 having a diameter greater than the diameter of the elongated body portion 252. As provided in FIG. 3, with the core member 250 in the first position, the proximal tapered surface 284 extends adjacent the distal opening of introducer 210 and the introducer 210 is maintained in the unexpanded configuration. In some implementations, the central lumen 212 of the introducer 210 includes a tapered opening surface 234 extending at a decreasing taper from the inner surface of the central lumen 212. The tapered opening surface 234 provides a tapered surface to ease withdraw of the expansion element 280 into the central lumen 212 of the introducer 210. As illustrated in FIG. 3, with the core member 250 in the first position, at least a portion of the proximal tapered surface 284 is located proximate and/or adjacent the tapered opening surface 234. In some implementations, the proximal tapered surface 284 and the tapered opening surface 234 extend at corresponding angles/directions.

In some implementations, the introducer 210 includes a plurality of slits 232 extending longitudinally along the introducer 210 and extending radially from the inner surface of the central lumen 212 through at least a portion of the wall thickness of the introducer 210. Separation/widening of each and/or at least one of the slits 232 facilitates expansion of the introducer 210 from the unexpanded configuration to the expanded configuration. In some implementations, the plurality of slits 232 extend along a portion of the length of the introducer 210. In further implementations, the plurality of slits 232 extend along the entirety of the length of the introducer 210. In some implementations, the slits 232 are evenly spaced around the circumference of the central lumen 212. It is contemplated that the introducer 210 can include at least one slit 232. In some implementations, the introducer 210 includes a number of slits ranging from one to 10. For example, the introducer 210 can include 1, 2, 3, 4, 5, 6, 7, 8, 9, or 10 slits 232. In a further implementation, the introducer 210 includes six slits 232 evenly spaced around the circumference of the central lumen 212 every 60-degrees.

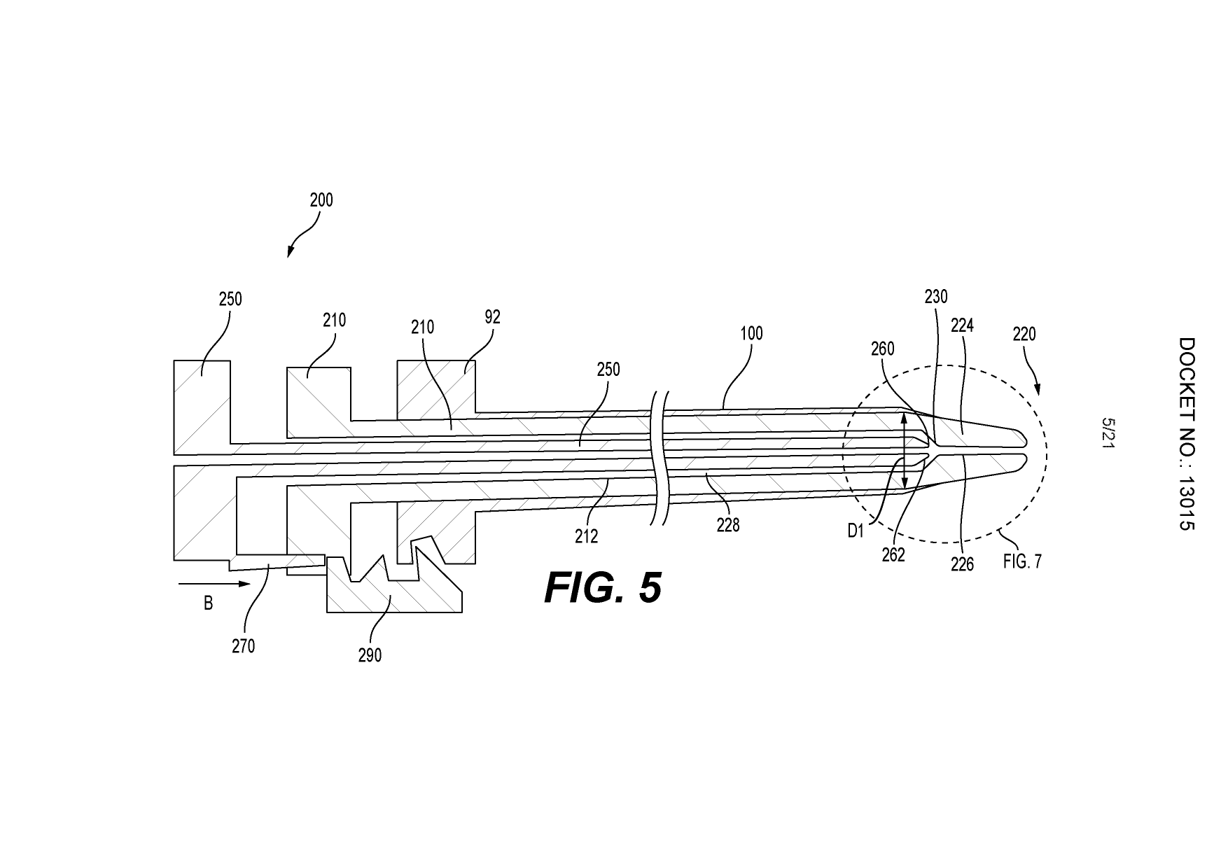

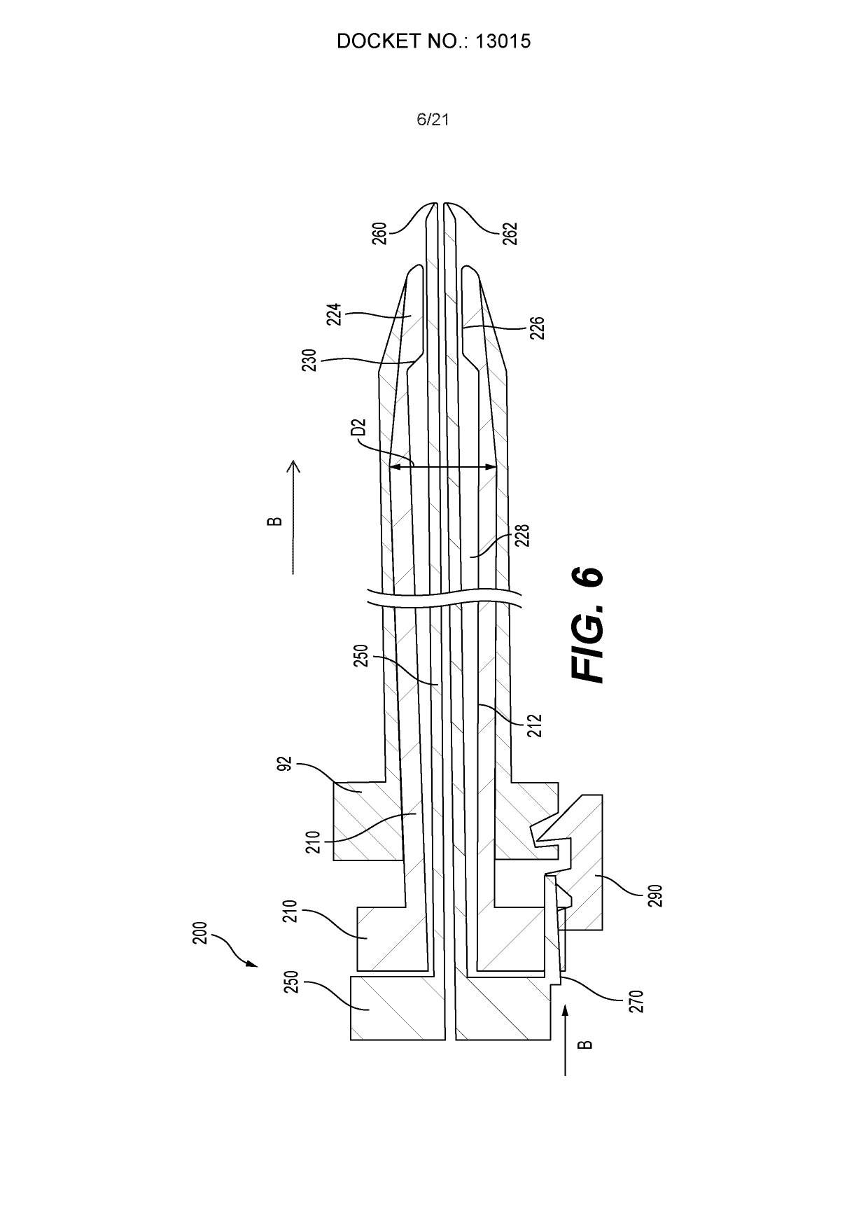

In some implementations and as described in more detail herein in reference to FIGS. 5-6, the introducer assembly 200 of FIGS. 3-4 includes an introducer locking mechanism for fixing the axial/longitudinal location of the introducer 210 with respect to the core member 250 and/or a sheath locking mechanism for fixing the axial/longitudinal location of the introducer 210 with respect to the sheath 100.

A method of dilating/expanding a sheath (for example, sheath 100) that can be used to deliver a medical device to a procedure site is described herein. In general, the introducer assembly 200 is positioned within the central lumen of the expandable sheath 100 and advanced to the treatment site. Once in position, the core member 250 is moved into the second position and the introducer 210 expanded. With the introducer 210/introducer assembly 200 in the expanded configuration, the introducer assembly 200 is withdrawn from the sheath 100, expanding a corresponding portion/length of the sheath 100 in response the outwardly directed radial force applied against the central lumen of the sheath 100 by the expanded introducer 210.

The method includes positioning the core member 250 within the central lumen 212 of the introducer 210 with the core member 250 in the first position such that the introducer 210 is in an unexpanded configuration. As illustrated in FIG. 3, in some implementations, when in the second position the core member 250 is advanced/located within the introducer 210 such that at least a portion of the expansion element 280 and/or the body portion 252 extends through a distal opening 218 of the introducer 210.

In the unexpanded configuration, the introducer 210 and core member 250 are advanced into the central lumen of the expandable sheath 100. In some implementations, the core member 250 is coupled to the introducer 210, before and/or after being advanced into the sheath 100, fixing the axial and/or rotational position of the core member with respect to the introducer 210 and preventing corresponding movement therebetween. For example, before the introducer assembly 200 is advanced into the sheath 100, the core member 250 is coupled to the introducer 210. In some implementations, the core member 250 is coupled to the introducer 210 using an introducer locking mechanism.

The combined core member 250, introducer 210 and sheath 100 are advanced into the patient’s blood vessel (for example, femoral artery) with the core member 250 in the first position with respect to the introducer 210.

The introducer 210 is then expanded by moving the core member 250 proximally within the central lumen 212 into the second position, thereby moving the introducer 210 from an unexpanded configuration to a corresponding expanded configuration in response to the outwardly directed radial force exerted on the central lumen 212 by the expansion element 280. In some implementations, before the introducer 210 is expanded, the introducer assembly 200 is coupled to the sheath 100 fixing the axial and/or rotational position of the introducer 210 with respect to the sheath 100 and preventing corresponding movement therebetween. For example, in some implementations, the introducer 210 is coupled to the proximal hub of the sheath 100 via a sheath locking mechanism 290. With the introducer 210 coupled to the sheath 100, the core member 250 is moved to the second position moving the introducer 210 from the unexpanded to the expanded configuration.

The sheath 100 is then expanded by moving the core member 250 and the introducer 210 together in the expanded configuration within the central lumen of the sheath 100. In some implementations, the expanded introducer assembly 200 is moved within the central lumen of the sheath 100 by withdrawing/moving introducer assembly 200 toward the proximal end of the sheath 100. As a result, contact between the expanded introducer 210 and the sheath 100 results in a corresponding radial expansion of the sheath 100. For example, the sheath 100 is expanded as the introducer assembly 200 including the core member 250 and introducer 210 together are removed from the sheath 100.

In some implementations, the sheath 100 is expanded by moving the core member 250 alone, for example, withdrawing/moving the core member 250 proximally, within the central lumen 212 of the introducer 210 while the introducer 210 remains within the central lumen of the sheath 100. As a result, contact between the expansion element 280 and the central lumen 212 of the introducer 210 provides an outwardly directed radial force on the central lumen 212 moving the introducer 210 from the unexpanded configuration to the expanded configuration resulting in a corresponding radial expansion along a length of the sheath 100. For example, the sheath 100 is expanded as the core member 250 is withdrawn from the introducer 210.

Expansion of the sheath 100 results in a corresponding expansion of the patient’s blood vessel. For example, a length of the blood vessel is expanded by expanding a corresponding length of the expandable sheath 100. With the sheath 100 expanded, the introducer 210 and core member 250 are withdrawn from the sheath 100 (and the blood vessel). The medical device 12 is advanced through the central lumen of the sheath 100 and beyond a distal opening of the sheath 100 to a treatment site. In some implementations, the medical device 12 is a prosthetic heart valve mounted in a radially crimped state on a delivery apparatus (for example, balloon catheter), and the method further comprises implanting the prosthetic heart valve at the treatment site within the patient.

FIGS. 5-7 illustrate an introducer assembly 200 according to another example. The introducer assembly 200 of FIGS. 5-7 includes structure and functions like the introducer assembly 200 of FIGS. 3-4. Like reference numbers are used to identify like features. The differences between the introducer assemblies of FIGS. 3-4 and FIGS. 5-7 are discussed in more detail herein.

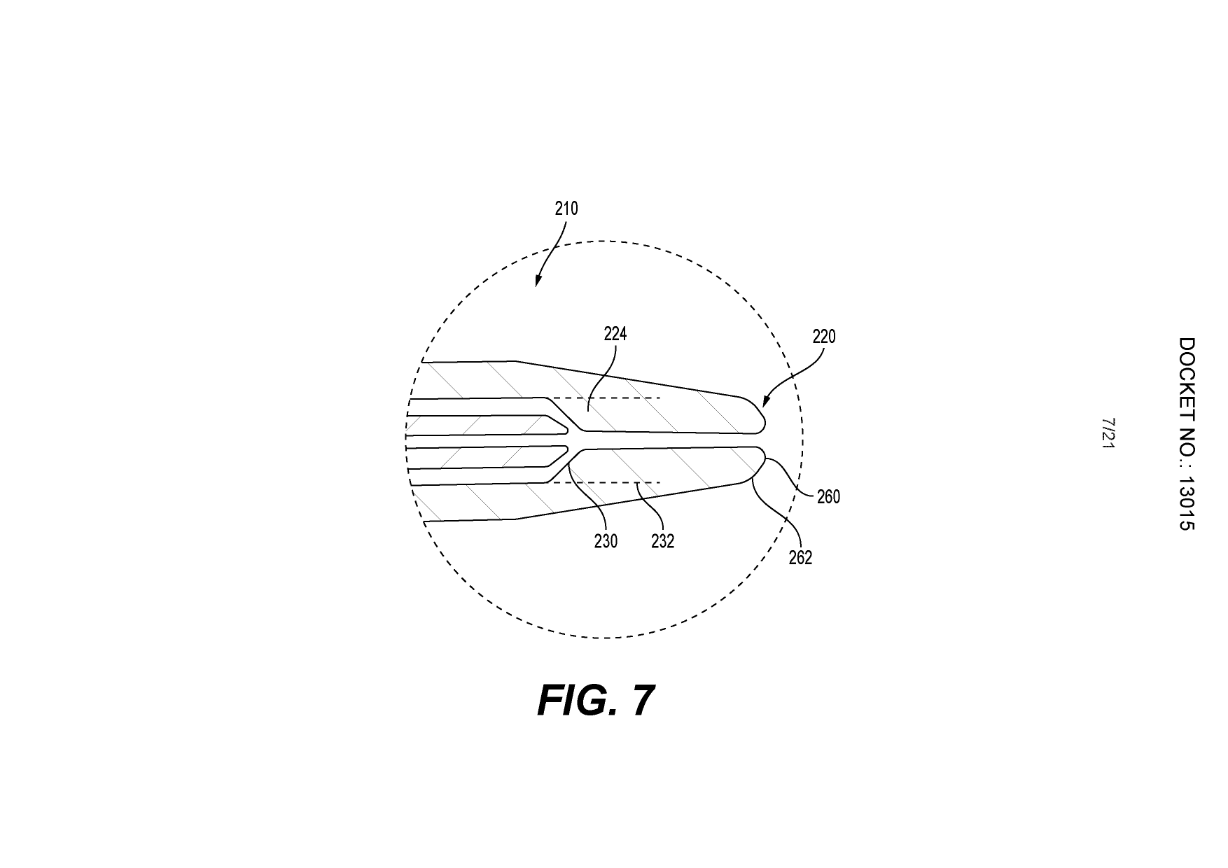

The introducer assembly 200 is sized and configured to be received within the expandable sheath 100 and radially expand from an unexpanded configuration (FIG. 5) to an expanded configuration (FIG. 6). With the introducer assembly 200 in the expanded configuration, it is withdrawn from the central lumen of the sheath 100 thereby expanding a corresponding portion/length the sheath 100. FIG. 5 is a side elevation cross-sectional view of the example introducer assembly 200 received within the expandable sheath 100 of FIG. 2 in the unexpanded configuration. FIG. 6 is a side elevation cross-sectional view of the introducer assembly 200 received within the expandable sheath 100 in an expanded configuration. FIG. 7 is an enlarged view of the distal end of the introducer assembly 200 of FIG. 5.

Like the introducer assembly 200 of FIGS. 3-4, the introducer assembly 200 of FIGS. 5-6 includes an elongated introducer 210 and an elongated core member 250. The core member 250 is received within the central lumen 212 of the introducer 210 which extends between a proximal opening 214 at the proximal end 216 of the introducer 210 and a distal opening 218 at the distal end 220 of the elongated introducer 210. The elongated core member 250 is sized and configured to be movably received within the central lumen 212 of the elongated introducer 210 between a first position (FIG. 5) and a second position (FIG. 6), resulting in the corresponding movement of the introducer 210 between the unexpanded and expanded configuration.

FIG. 5 illustrates the core member 250 received within the central lumen 212 of the introducer 210 in a first position where the introducer 210/introducer assembly 200 is in a corresponding unexpanded configuration having a first outer diameter (D1). FIG. 6 illustrates the core member 250 in a second position where the introducer 210 is in a corresponding expanded configuration having a second, larger, outer diameter (D2).

As illustrated in FIGS. 5-7, the introducer 210 includes an inner shoulder 224 extending radially inward from the inner surface of the introducer 210 forming the central lumen 212. In some implementations, the inner shoulder 224 extends longitudinally from the distal end 220 of the introducer 210 in a direction toward the proximal end 216 of the introducer 210. When the introducer 210 is in the unexpanded configuration, the inner shoulder 224 defines a reduced diameter portion 226 of the central lumen 212, the reduced diameter portion 226 having a diameter less than the diameter of the main diameter portion 228 of the central lumen 212. In some implementations, the inner shoulder 224 includes a proximal tapered surface 230 extending between the main diameter portion 228 and the reduced diameter portion 226. The proximal tapered surface 230 facilitates sliding movement between the core member 250 and the inner shoulder 224 as the core member 250 is moved distally during introducer 210 expansion.

When the core member 250 is in the first position, the distal end 260 of the core member 250 is positioned proximal of the inner shoulder 224. At least a portion of the introducer 210 is configured to expand to the expanded configuration due to the outwardly directed radial force exerted on the inner shoulder 224 by the core member 250 as it moved between the first and second positions. For example, as the core member 250 moves from the first position to the second position, the core member is moved in a distal direction (direction B) within the central lumen 212 of the introducer 210 and engages the inner shoulder 224. In some implementations, when the core member 250 is in the second position, the core member 250 is positioned adjacent the inner shoulder 224 such that contact between the core member 250 and the inner shoulder 224 provides the outwardly directed radial force on the introducer 210 expanding the introducer and moving the introducer 210 from the unexpanded configuration to the expanded configuration.

As illustrated in FIG. 7, in some implementations, the introducer 210 includes a plurality of slits 232 extending longitudinally along the inner shoulder 224. The slits 232 extending radially from the inner surface of the central lumen 122 through at least a portion of the wall thickness of the introducer 210/inner shoulder 224. Separation/widening of at least one of the slits facilitates expansion of the introducer 210 from the unexpanded configuration to the expanded configuration. In some implementations, the slits 232 are located adjacent the distal end 220 along a portion of the inner shoulder 224 such that the slits 232 facilitate expansion of a distal end portion of the introducer 210. In some examples, the slits 232 extend along the entire length of the inner shoulder 224. In some implementations, the slits 232 are evenly spaced around the circumference of the central lumen 212/inner shoulder 224. It is contemplated that the introducer 210 can include at least one slit 232. It is contemplated, that in some implementations, the introducer 210 includes a number of slits ranging from one to 10. For example, the introducer 210 can include 1, 2, 3, 4, 5, 6, 7, 8, 9, or 10 slits 232. In a further implementation, the introducer 210 includes six slits 232 evenly spaced around the circumference of the central lumen 212 every 60-degrees.

As illustrated in FIGS. 5 and 6, the core member 250 includes a central lumen 256 extending between a proximal end 258 and distal end 260 of the core member 250. In some implementations, the central lumen 256 is sized and configurated to receive a guide wire. In some implementations, the core member 250 has a constant cross-sectional shape along a majority of the length of the core member 250. In further implementations, the core member 250 has a generally constant outer diameter along a length of the core member. For example, the core member 250 can have a constant outer diameter along the entire length of the core member 250 up to the distal tapered surface 362. As provided in FIG. 7, the core member 250 includes a distal tapered surface 262. In some implementations, the distal tapered surface 262 extends from the distal end 260 of the core member 250 to facilitate advancement of the core member 250 along the inner shoulder 224.

In some implementations, as illustrated in FIGS. 5 and 6, the introducer assembly 200 includes an introducer locking mechanism 270 for fixing the position of the introducer 210 with respect to the core member 250. For example, the introducer locking mechanism 270 is used to fix the introducer 210 and core member 250 during insertion of the introducer assembly 200 into the sheath 100. In some implementations, the introducer locking mechanism 270 fixes the axial/longitudinal location of the introducer 210 with respect to the core member 250. In some implementations, the introducer locking mechanism 270 also fixes the rotational position of the introducer 210 with respect to the core member 250. In some implementations, the introducer locking mechanism 270 fixes the location of the core member 250 at various axial positions such that the expansion element 280 is located positions corresponding to the desired outer diameter of the introducer 210. In some examples, as illustrated in FIG. 5, with the introducer locking mechanism 270 in a locked position, the position/location of the core member 250 is maintained in the first position where the proximal end 258 of the core member 250 is spaced from the proximal end 216 of the introducer 210, and the distal end 260 of the core member 250 is spaced from and/or not engaged with the inner shoulder 224. With the core member 250 fixed in the first position by the introducer locking mechanism 270, the introducer assembly 200 can be advanced into the sheath 100 in unexpended configuration. Once in position, the introducer locking mechanism 270 and can be disengaged/moved to an unlocked position and the core member 250 is free to move within the introducer 210 to the second position, thereby expanding the introducer 210. In some implementations, the introducer locking mechanism 270 includes a projection/tooth that engages a corresponding recess/opening/catch provided on the introducer 210.

In some implementations, the core member 250 and/or introducer 210 can include a marker for indicating the corresponding to the diameter of the introducer 210 at the expansion element 280 based on the relative position between the core member 250 and the introducer 210. The marker allows the user to measure the diameter of the introducer 210 while the distal end of the introducer 210 is positioned in the patient’s vasculature. In some implementations, the marker is provided as a visual indicia on the outer surface of the core member 250 and/or introducer 210. In some implementations, the marker is a radio-opaque marker visual under radio ﬂuoroscopy.

In further implementations, the introducer assembly 200 includes a sheath locking mechanism 290 for fixing the axial/longitudinal location of the introducer 210 with respect to the sheath 100. In some implementations, the sheath locking mechanism 290 fixes the axial and/or rotational position of the introducer 210 with respect to the sheath 100. For example, as illustrated in FIG. 5, the sheath locking mechanism 290 maintains the position of the introducer 210 with respect to the sheath 100 while the core member 250 is moved between the first and second position (resulting in the corresponding movement of the introducer 210 between the unexpanded and expanded configuration). In some implementations, as illustrated in FIG. 5, the sheath locking mechanism 290 includes a projection/tooth that engages a corresponding recess/opening/catch provided on the introducer 210 and/or the sheath hub. Once the core member 250 is in the second position, and the introducer 210 in the expanded configuration, the sheath locking mechanism 290 can be released and the entire introducer assembly 200 is free to be withdrawn from the sheath 100 in the expanded configuration. In some implementations, advancing the core member 250 within the introducer 210 to the second position causes the sheath locking mechanism 290 to release from the sheath 100.

A method of dilating/expanding a sheath (for example, sheath 100) using the introducer assembly 200 of FIGS. 5-7 is described herein. The method includes positioning the core member 250 within the central lumen 212 of the introducer 210 in a first position.

In some implementations, before insertion of the introducer assembly 200 into the sheath 100, the core member 250 is coupled to the introducer 210 fixing the position of the core member 250 with respect to the introducer 210. For example, the core member 250 is coupled to the introducer 210 by engaging the introducer locking mechanism 270. The combined/coupled core member 250 and introducer 210 are advanced into the central lumen of the expandable sheath 100 in the unexpanded configuration.

In some implementations, the introducer 210 and/or core member 250 are coupled to the expandable sheath 100 fixing the position of the unexpanded introducer assembly 200 with respect to the sheath 100. For example, the introducer assembly 200 is coupled to the sheath 100 by engaging the sheath locking mechanism 290.

The combined core member 250, introducer 210 and sheath 100 are advanced into the patient’s blood vessel (for example, femoral artery) with the core member 250 in the first position with respect to the introducer 210.

The core member 250 is uncoupled from the introducer 210 such that the core member 250 is capable of freely moving (for example, axially and rotationally) with respect to the introducer 210. For example, the core member 250 is uncoupled from the introducer 210 by disengaging the introducer locking mechanism 270.

The introducer 210 is then expanded by moving the core member 250 from the first position, where the distal end 260 of the core member 250 is positioned proximal of the inner shoulder 224, into the second position, where the core member 250 is advanced with the central lumen 212 and in contact with the inner shoulder 224. As a result, the introducer 210 moves from the unexpanded configuration to the expanded configuration due to the outwardly directed radial force exerted on the inner shoulder 224 by the core member 250.

The introducer 210 and/or core member 250 is then uncoupled from the expandable sheath 100. For example, the introducer assembly 200 is uncoupled from the sheath 100 by disengaging the sheath locking mechanism 290.

The sheath 100 is expanded by moving the core member 250 and the introducer 210 together in the expanded configuration within the central lumen of the sheath 100. In some implementations, the expanded introducer assembly 200 is moved within the central lumen by withdrawing introducer assembly 200 from the sheath 100 and/or moving the introducer assembly 200 toward the proximal end of the sheath 100. As a result, contact between the expanded introducer 210 and the sheath 100 results in a corresponding radial expansion of the sheath 100. For example, the sheath 100 is expanded as the introducer assembly 200 including the core member 250 and introducer 210 together are removed from the sheath 100.

Expansion of the sheath 100 results in a corresponding expansion of the patient’s blood vessel. For example, a length of the blood vessel is expanded by expanding a corresponding length of the expandable sheath 100. With the sheath 100 expanded, the introducer 210 and core member 250 are withdrawn from the sheath 100 and the blood vessel. The medical device 12 is advanced through the central lumen of the sheath 100 and beyond a distal opening of the sheath 100 to a treatment site. In some implementations, the medical device 12 is a prosthetic heart valve mounted in a radially crimped state on a delivery apparatus (for example, balloon catheter), and the method further comprises implanting the prosthetic heart valve at the treatment site within the patient.

FIG. 8 illustrates the expandable sheath 100 of FIG. 1 used with the introducer assembly 200 in greater detail. With reference to FIG. 8, the sheath 100 can have a natural, unexpanded outer diameter D1. Examples of such an expandable sheath 100 is described in PCT/US2021/054788 and PCT/US2022/038481, the disclosures of which area herein incorporated by reference. In certain implementations, the expandable sheath 100 can comprise a plurality of coaxial layers extending along at least a portion of the length L of the sheath (FIG. 2). For example, with reference to FIG. 9, the expandable sheath 100 can include a first layer 102 (also referred to as an inner layer or an inner liner), a second layer 104 (also referred to as a braided layer) disposed around and radially outward of the first layer 102, a third layer 106 disposed around and radially outward of the second layer 104, and a fourth layer 108 (also referred to as an outer layer or an outer liner or polymeric layer) disposed around and radially outward of the third layer 106. In the illustrated configuration, the inner layer 102 (liner) can define the central lumen 112 of the sheath extending along a central axis 114.

Referring to FIG. 8, when the sheath 100 is in an unexpanded state, the inner layer (liner) 102 and/or the outer layer (liner) 108 can form longitudinally-extending folds or pleats or creases such that the surface of the sheath comprises a plurality of ridges 126 (also referred to herein as “folds” or “pleats”). The ridges 126 can be circumferentially spaced apart from each other by longitudinally-extending valleys 128. When the sheath expands beyond its natural (initial) diameter D1, the ridges 126 and the valleys 128 can level out or be taken up as the surface radially expands and the circumference increases, as described herein. When the sheath collapses back to its natural diameter (or, in other words, returns to its unexpanded state), the ridges 126 and valleys 128 can reform.

In some implementations, the inner layer (liner) 102 and/or the outer layer (liner) 108 can comprise a relatively thin layer of polymeric material. For example, in some implementations, the thickness of the inner layer 102 can be from 0.01 mm to 0.5 mm, 0.02 mm to 0.4 mm, or 0.03 mm to 0.25 mm. In certain implementations, the thickness of the outer layer 108 can be from 0.01 mm to 0.5 mm, 0.02 mm to 0.4 mm, or 0.03 mm to 0.25 mm. In yet some implementations, the inner layer 102 and the outer liner 108 can comprise at least one polymer layer. In some implementations, the inner layer 102 and the outer liner 108 each can comprise two or more layers of polymeric material.

In some implementations, the inner layer 102 and/or the outer layer 108 can comprise a lubricious, low-friction, and/or relatively non-elastic material. In some implementations, the inner layer 102 and/or the outer layer 108 can comprise a polymeric material having a modulus of elasticity of 400 MPa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such a low coefficient of friction materials can facilitate the passage of the medical device 12 (for example, a prosthetic device) through the central lumen 112. Some suitable materials for the inner layer 102 and outer layer 108 can include polytetrafluoroethylene (PTFE), expanded polytetrafluoroethylene (ePTFE), ethylene tetrafluoroethylene (ETFE), nylon, polyethylene (such as, for example, low-density polyethylene (LDPE), high density polyethylene (HDPE)), polyether block amide (for example, Pebax), bi-oriented polypropylene, cast polypropylene, thermoplastic polyurethane, and/or combinations of any of the above. Some implementations of a sheath 100 can include an additional lubricious liner on the inner surface of the inner layer 102. Examples of such suitable lubricious liners include materials that can further reduce the coefficient of friction of the inner layer 102, such as PTFE, polyethylene (such as, for example, HMWPE, UHMWPE, LDPE, HDPE), polyvinylidine fluoride, and combinations thereof. Suitable materials for a lubricious liner also include some materials desirably having a coefficient of friction of 0.1 or less.

Additionally, some implementations of the sheath 100 can include an exterior hydrophilic coating on the outer surface of the outer layer (liner) 108. Such a hydrophilic coating can facilitate the insertion of the sheath 100 into a patient’s vessel, reducing potential damage. Examples of suitable hydrophilic coatings include the Harmony^{TM} Advanced Lubricity Coatings and other Advanced Hydrophilic Coatings available from SurModics, Inc., Eden Prairie, MN. DSM medical coatings (available from Koninklijke DSM NV, Heerlen, the Netherlands), as well as other hydrophilic coatings (for example, PTFE, polyethylene, polyvinylidene fluoride), are also suitable for use with the sheath 100. Such hydrophilic coatings may also be included on the inner surface of the inner layer 102 to reduce friction between the sheath and the delivery system, thereby facilitating the use and improving safety. In some implementations, a hydrophobic coating, such as Perylene, may be used on the outer surface of the outer layer 108 or the inner surface of the inner layer 102 in order to reduce friction.

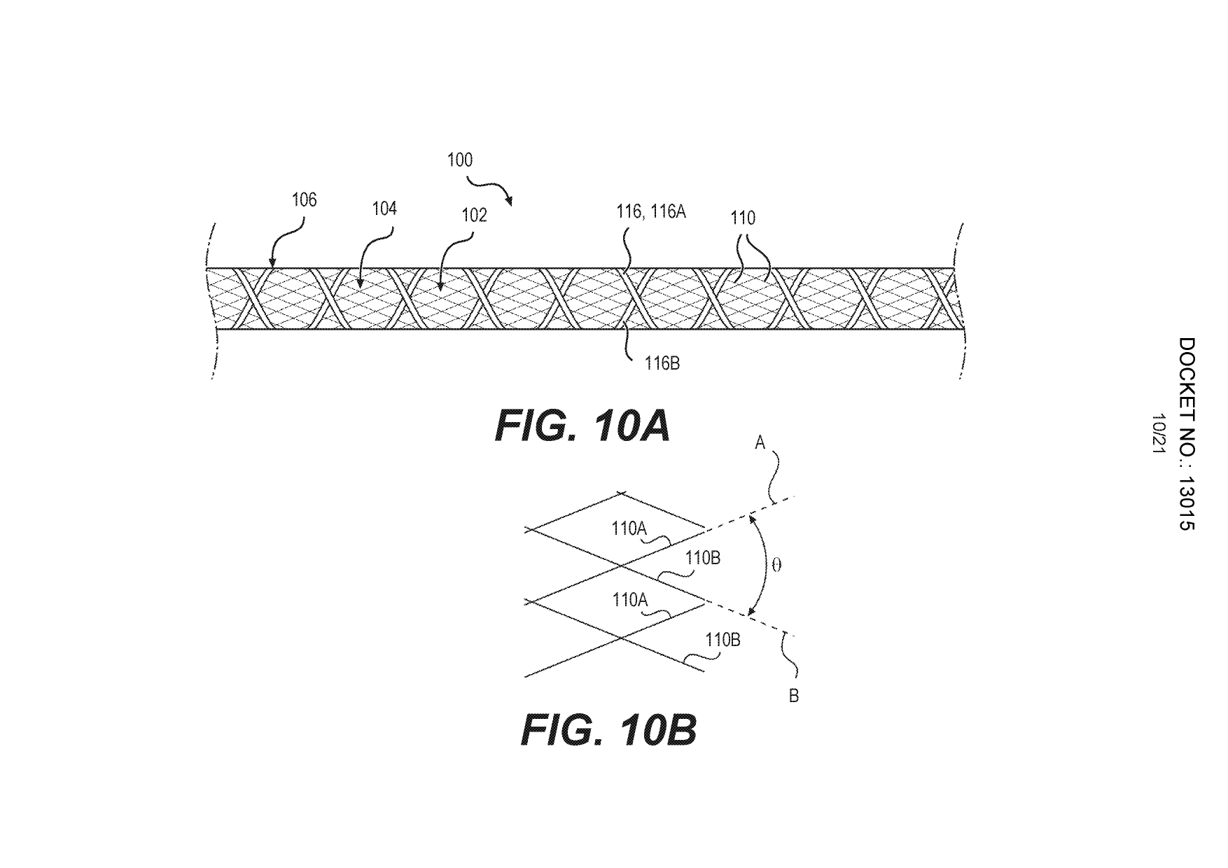

In some implementations, the second layer 104 can be braided (referred to herein as a braided layer 104). FIGS. 10A and 10B illustrate the sheath 100 with the outer layer 108 removed to expose the third layer 106. With reference to FIGS. 10A and 10B, the braided layer 104 can comprise a plurality of members or filaments 110 (for example, metallic or synthetic wires or fibers) braided together. The braided layer 104 can have any desired number of filaments 110, which can be oriented and braided together along any suitable number of axes. For example, with reference to FIG. 10B, the filaments 110 can include a first set of filaments 110A oriented parallel to a first axis A and a second set of filaments 110B oriented parallel to a second axis B. The first set of filaments 110A and second set of filaments 110B can be braided together in a biaxial braid such that the first set of filaments 110A oriented along axis A form an angle θ with the second set of filaments 110B oriented along axis B. In some implementations, the angle θ can be from 5° to 70°, 10° to 60°, 10° to 50°, or 10° to 45°. In the illustrated implementation, the angle θ is 45°; however, it is understood that this is for exemplary purposes only and is not limiting. In some implementations, the filaments 110 can also be oriented along three axes and braided in a triaxial braid or oriented along any number of axes and braided in any suitable braid pattern.

The braided layer 104 can extend along substantially the entire length L of the sheath 100, or alternatively, it can extend only along a portion of the length of the sheath. In some implementations, the filaments 110 can be wires made from metal (for example, Nitinol, stainless steel, etc.) or any of various polymers or polymer composite materials, such as carbon fiber. In some implementations, the filaments 110 can be round and can have a diameter of from 0.01 mm to 0.5 mm, 0.03 mm to 0.4 mm, or 0.05 mm to 0.25 mm. In some implementations, the filaments 110 can have a flat cross-section with dimensions of 0.01 mm x 0.01 mm to 0.5 mm x 0.5 mm, or 0.05 mm x 0.05 mm to 0.25 mm x 0.25 mm. In one implementation, filaments 110 having a flat cross-section can have dimensions of 0.1 mm x 0.2 mm. However, other geometries and sizes are also suitable for certain implementations. If a braided wire is used, the braid density can be varied. Some implementations have a braid density of from ten picks per inch to eighty picks per inch and can include eight wires, sixteen wires, or up to fifty-two wires in various braid patterns. In some implementations, the second layer 104 can be laser cut from a tube, or laser-cut, stamped, punched, etc., from sheet stock and rolled into a tubular configuration. Layer 104 can also be woven or knitted, as desired. In some implementations, the braided layer can have a weave pattern of, for example, 1 x 1 (one over, one under), 2 x 2 (two over, two under), or 2 x 1 (two over, one under).

A braided layer 104 (also referred to herein as the second layer 104) can comprise any known in the art material that can be provided for the desired expansion of the sheath. For example, and without limitation, the braided layer 104 can comprise Nitinol or some other shape memory metal or material that can exhibit superelastic properties. In such implementations, these materials can have the advantage of allowing for austenitic finishing (AF) at a certain temperature. For example, a nitinol braided layer having AF at 15 degrees Celsius or less allows for its use in relatively cold operating rooms while still exhibiting superelastic properties. In some implementations, the materials used to form the braided layer 104 can exhibit superelastic properties at temperatures at or above about 15 degrees Celsius.

The third layer 106 can be a resilient, elastic layer 106 (also referred to as an elastic material layer). In certain implementations, the elastic layer 106 can be configured to apply force to the underlying inner layer 102 and second layer 104 in a radial direction (for example, toward the central axis 114 of the sheath) when the sheath expands beyond its natural diameter by the passage of the delivery apparatus through the sheath 100. Stated differently, the elastic layer 106 can be configured to apply encircling pressure to the layers of the sheath beneath the elastic layer 106 to counteract expansion of the sheath 100. The radially inwardly directed force is sufficient to cause the sheath 100 to collapse radially back to its unexpanded state after the delivery apparatus is passed through the sheath 100. It is understood, however, that elastic layer 106 can be optional. And also described herein are the implementations where this elastic layer 106 is not present, while all other layers described herein are. It is also understood that this description includes all various combinations of the layers, and unless it is stated otherwise, some of the described herein layers (liners) can be present while others can be absent. In some implementations, and as shown herein, additional layers can also be present.

In the illustrated implementation, the elastic layer 106 can comprise one or more members configured as strands, ribbons, or bands 116 helically wrapped around the braided layer 104. For example, in the illustrated implementation, the elastic layer 106 comprises two elastic bands, 116A and 116B, wrapped around the braided layer with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some implementations, the elastic layer 106 can comprise an elastomeric material having a modulus of elasticity of 200 MPa or less. In some implementations, the elastic layer 106 can comprise a material exhibiting an elongation to break of 200% or greater or an elongation to break of 400% or greater. The elastic layer 106 can also take other forms, such as a tubular layer comprising an elastomeric material, a mesh, a shrinkable polymer layer such as a heat-shrink tubing layer, etc. In lieu of, or in addition to, the elastic layer 106, the sheath 100 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In some implementations, the elastic layer 106 can also be radially outward of the polymeric layer 108.

In certain implementations, one or both of the inner layer 102 and/or the outer layer 108 can be configured to resist axial elongation of the sheath 100 when the sheath expands. More particularly, one or both of the inner layer 102 and/or the outer layer 108 can resist stretching against longitudinal forces caused by friction between a medical device 12 and the inner surface of the sheath 100 such that the length L remains substantially constant as the sheath 100 expands and contracts. As used herein with reference to the length L of the sheath 100, the term “substantially constant” means that the length L of the sheath 100 increases by not more than 1%, by not more than 5%, by not more than 10%, by not more than 15%, or by not more than 20%. Meanwhile, with reference to FIG. 10B, the filaments 110A and 110B of the braided layer 104 can be allowed to move angularly relative to each other such that the angle θ changes as the sheath 100 expands and contracts. This, in combination with the longitudinal folds/ridges 126 in the inner layer 102 and outer layer 108, can allow the central lumen 112 of the sheath 100 to expand as a medical device 12 is advanced through it.

For example, in some implementations, the inner layer 102 and the outer layer 108 can be heat-bonded during the manufacturing process, such that the braided layer 104 and the elastic layer 106 are encapsulated between the inner layer 102 and outer layer 108. More specifically, in certain implementations, the inner layer 102 and the outer layer 108 can be adhered to each other through the spaces between the filaments 110 of the braided layer 104 and/or the spaces between the elastic bands 116. The inner layer 102 and outer layer 108 can also be bonded or adhered together at the proximal and/or distal ends of the sheath 100. In certain implementations, the inner layer 102 and outer layer 108 are not adhered to the filaments 110. This can allow the filaments 110 to move angularly relative to each other and relative to the inner layer 102 and outer layer 108, allowing the diameter of the braided layer 104, and thereby the diameter of the sheath 100, to increase or decrease. As the angle θ between the filaments 110A and 110B changes, the length of the braided layer 104 can also change. For example, as the angle θ increases, the braided layer 104 can foreshorten, and as the angle θ decreases, the braided layer 104 can lengthen to the extent permitted by the areas where the inner layer 102 and outer layer 108 are bonded. However, because the braided layer 104 is not adhered to the inner layer 102 and outer layer 108, the change in length of the braided layer 104 that accompanies a change in the angle θ between the filaments 110A and 110B does not result in a significant change in the length L of the sheath 100.

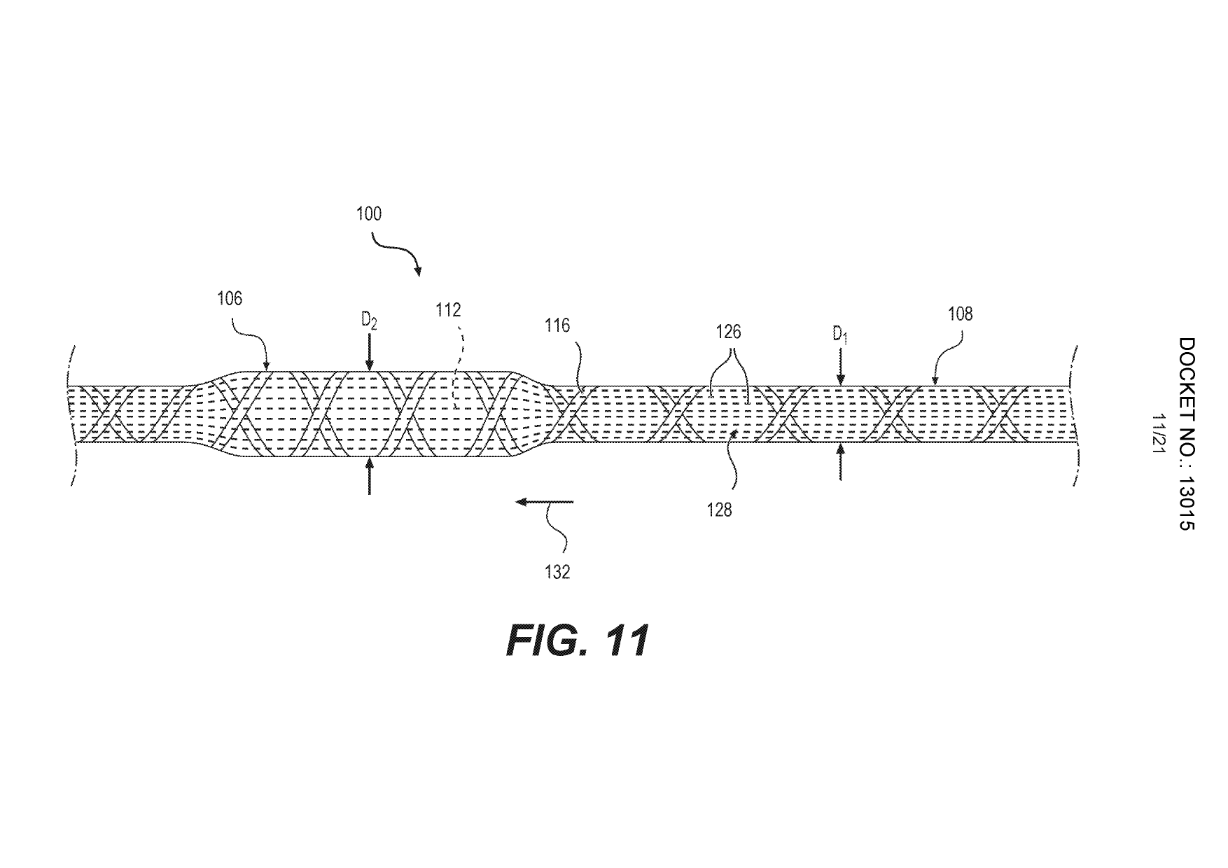

FIG. 11 illustrates a local radial expansion of the sheath 100 as a medical device 12 is passed through the sheath in the direction of arrow 132 (for example, distally). As the medical device 12 is advanced through the sheath 100, the sheath can resiliently locally expand to a second diameter D2 that corresponds to the size or diameter of the medical device 12. As medical device 12 is advanced through the sheath 100, the medical device 12 can apply longitudinal force to the sheath in the direction of motion by virtue of the frictional contact between the medical device 12 and the inner surface of the sheath 100. However, as noted herein, the inner layer (liner) 102 and/or the outer layer (liner) 108 can resist axial elongation such that the length L of the sheath 100 remains constant or substantially constant. This can reduce or prevent the braided layer 104 from lengthening, and thereby constricting the central lumen 112.

Meanwhile, the angle θ between the filaments 110A and 110B can increase as the sheath expands to the second diameter D2 to accommodate the medical device 12 (for example, a prosthetic heart valve). This can cause the braided layer 104 to be foreshortened. However, because the filaments 110 are not engaged or adhered to the inner layer 102 or outer layer 108, the shortening of the braided layer 104 attendant to an increase in the angle θ does not affect the overall length L of the sheath. Moreover, because of the longitudinally-extending folds/ridges 126 formed in the inner layer 102 and outer layer 108, the inner layer 102 and outer layer 108 can expand to the second diameter D2 without rupturing, despite being relatively thin and relatively non-elastic. In this manner, the sheath 100 can resiliently expand from its natural diameter D1 to a second diameter D2 that is larger than the diameter D1 as a medical device 12 is advanced through the sheath 100, without lengthening and without constricting. Thus, the force required to push the medical device 12 through the sheath 100 is significantly reduced.

Additionally, because of the radial force applied by the elastic layer 106, the radial expansion of the sheath 100 can be localized to the specific portion of the sheath 100 occupied by the medical device 12. For example, with reference to FIG. 11, as the medical device 12 moves distally through the sheath 100, the portion of the sheath 100 immediately proximal to the medical device 12 can radially collapse back to the initial diameter D1 under the influence of the elastic layer 106. The inner layer 102 and outer layer 108 can also buckle as the circumference of the sheath 100 is reduced, causing the ridges 126 and the valleys 128 to reform. This can reduce the size of the sheath 100 required to introduce a medical device 12 of a given size. Additionally, the temporary, localized nature of the expansion can reduce trauma to the blood vessel into which the sheath 100 is inserted, along with the surrounding tissue, because only the portion of the sheath 100 occupied by the medical device 12 expands beyond the sheath’s 100 natural diameter and the sheath 100 collapses back to the initial diameter once the medical device 12 has passed. This limits the amount of tissue that must be stretched in order to introduce the medical device 12 and the amount of time for which a given portion of the vessel must be dilated.

In addition to the advantages above, the expandable sheath implementations described herein can provide surprisingly superior performance relative to known introducer sheaths. For example, it is possible to use a sheath 100 configured as described herein to deliver a medical device 12 (for example a prosthetic heart valve) having a diameter that is two times larger, 2.5 times larger, or even three times larger than the natural outer diameter of the sheath 100. For example, in one implementation, a crimped prosthetic heart valve having a diameter of 7.2 mm was successfully advanced through a sheath 100 configured as described herein and having a natural outer diameter of 3.7 mm. As the prosthetic heart valve was advanced through the sheath 100, the outer diameter of the portion of the sheath 100 occupied by the prosthetic heart valve increased to 8 mm. In other words, it was possible to advance a medical device 12 having a diameter more than two times the outer diameter of the sheath through the sheath 100, during which the outer diameter of the sheath 100 resiliently increased by 216%. In some examples, a sheath 100 with an initial or natural outer diameter of 4.5 mm to 5 mm can be configured to expand to an outer diameter of 8 mm to 9 mm.

In some implementations, the sheath 100 may optionally include inner layer 102 without outer layer 108, or outer layer 108 without inner layer 102, depending upon the particular characteristics desired.

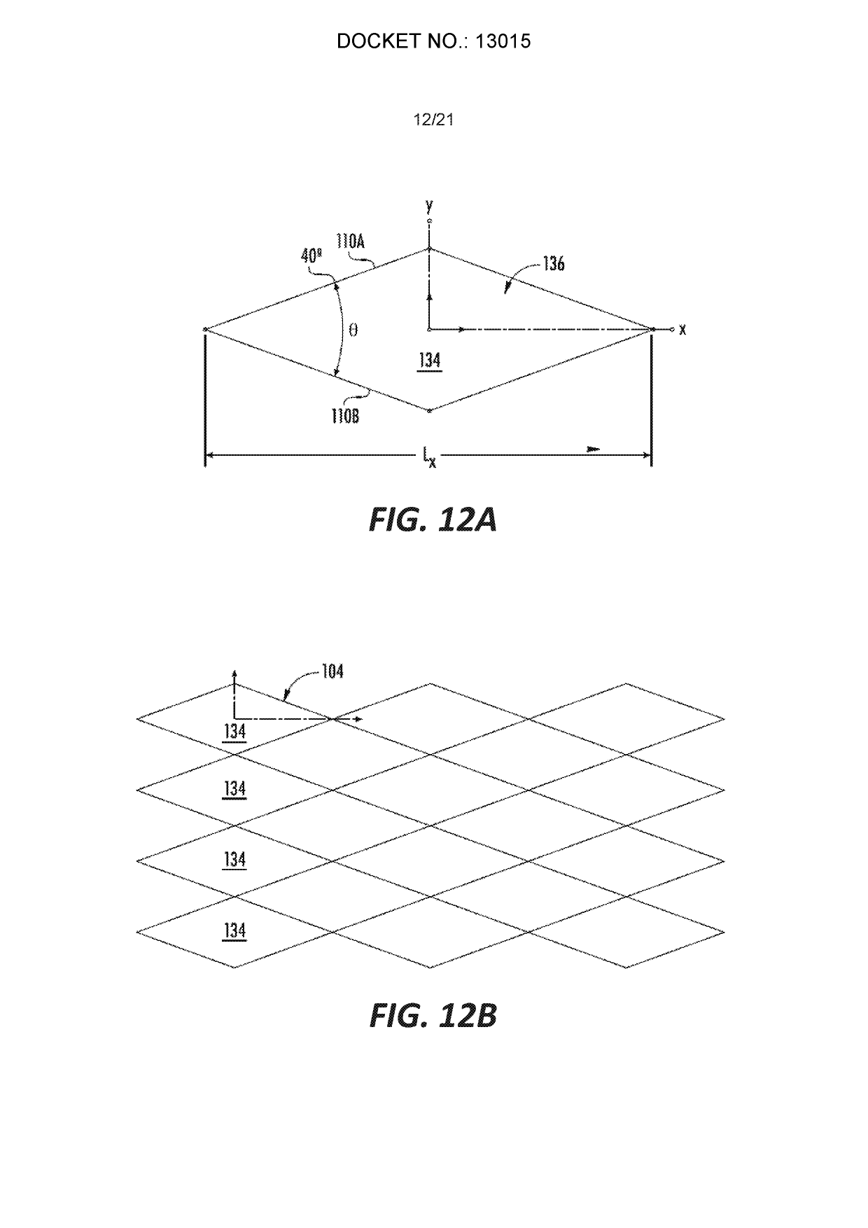

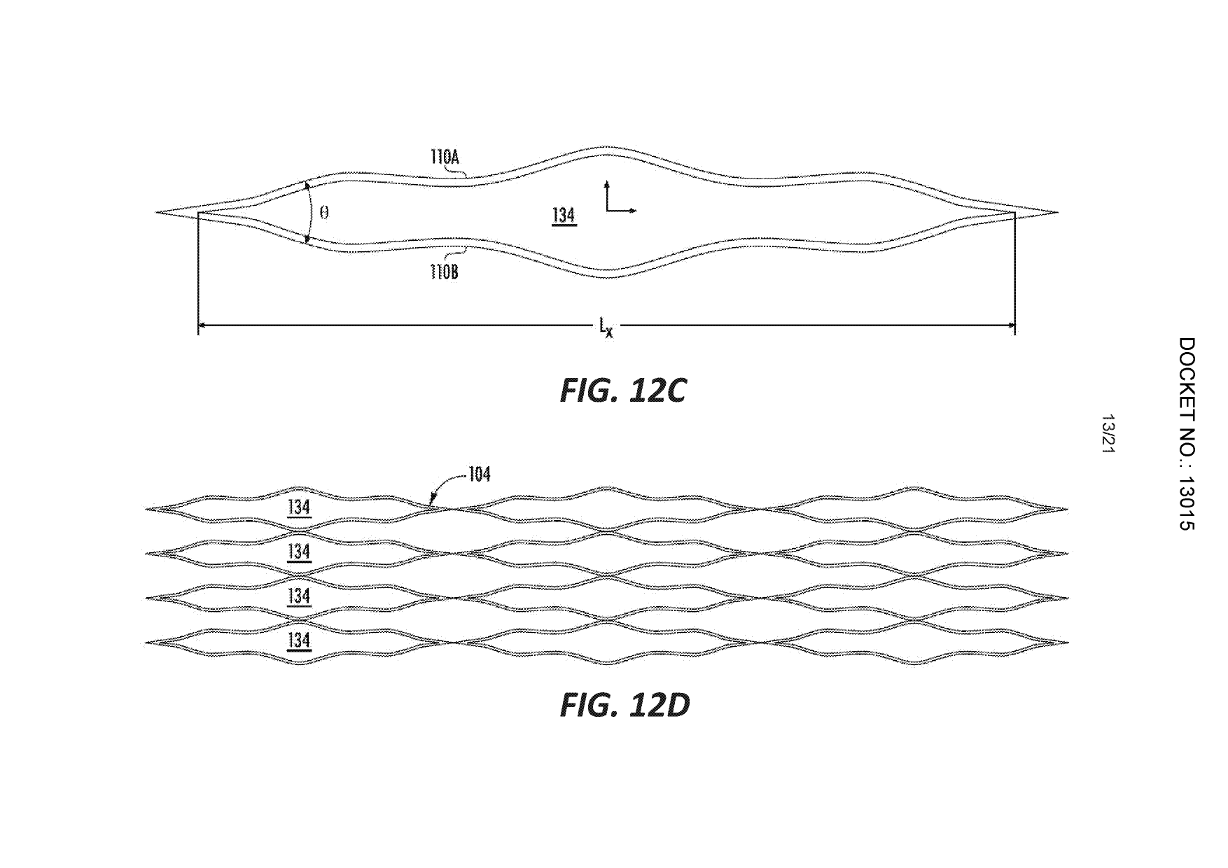

FIGS. 12A-12D illustrate some implementations of the braided layer 104 in which the filaments 110 are configured to buckle. For example, FIG. 12A illustrates a unit cell 134 of the braided layer 104 in a configuration corresponding to the braided layer 104 in a fully expanded state. For example, the expanded state illustrated in FIG. 12A can correspond to the diameter D2 described above and/or a diameter of the braided layer during the initial construction of the sheath 100 before the sheath is radially collapsed to its functional design diameter D1. The angle θ between the filaments 110A and 110B can be, for example, 40°, and the unit cell 134 can have a length Lx along the x-direction (note Cartesian coordinate axes shown). FIG. 12B illustrates a portion of the braided layer 104, including an array of unit cells 134 in the expanded state.

In the illustrated implementations, the braided layer 104 is disposed between the polymeric inner layer 102 and polymeric outer layer 108, as described herein. For example, the polymeric inner layer 102 and polymeric outer layer 108 can be adhered or laminated to each other at the ends of the sheath 100 and/or between the filaments 110 in the open spaces 136 defined by the unit cells 134. Thus, with reference to FIGS. 12C and 12D, when the sheath 100 is radially collapsed to its functional diameter D1, the diameter of the braided layer 104 can decrease as the angle θ decreases. However, the bonded polymeric inner layer 102 and polymeric outer layer 108 can constrain or prevent the braided layer 104 from lengthening as it radially collapses. This can cause the filaments 110 to resiliently buckle in the axial direction, as shown in FIGS. 12C and 12D. The degree of buckling can be such that the length Lx of the unit cells 134 is the same, or substantially the same, between the collapsed and fully expanded diameters of the sheath 100. This means that the overall length of the braided layer 104 can remain constant, or substantially constant, between the natural diameter D1 of the sheath and the expanded diameter D2. As the sheath 100 expands from its initial diameter D1 during passage of a medical device 12, the filaments 110 can straighten as the buckling is relieved, and the sheath 100 can radially expand. As the medical device 12 passes through the sheath 100, the braided layer 104 can be urged back to the initial diameter D1 by the elastic layer 106, if present, and the filaments 110 can resiliently buckle again. Using the configuration of FIGS. 12A-12C, it is also possible to accommodate a medical device 12 having a diameter that is two times larger, 2.5 times larger, or even three times larger than the natural outer diameter D1 of the sheath 100.

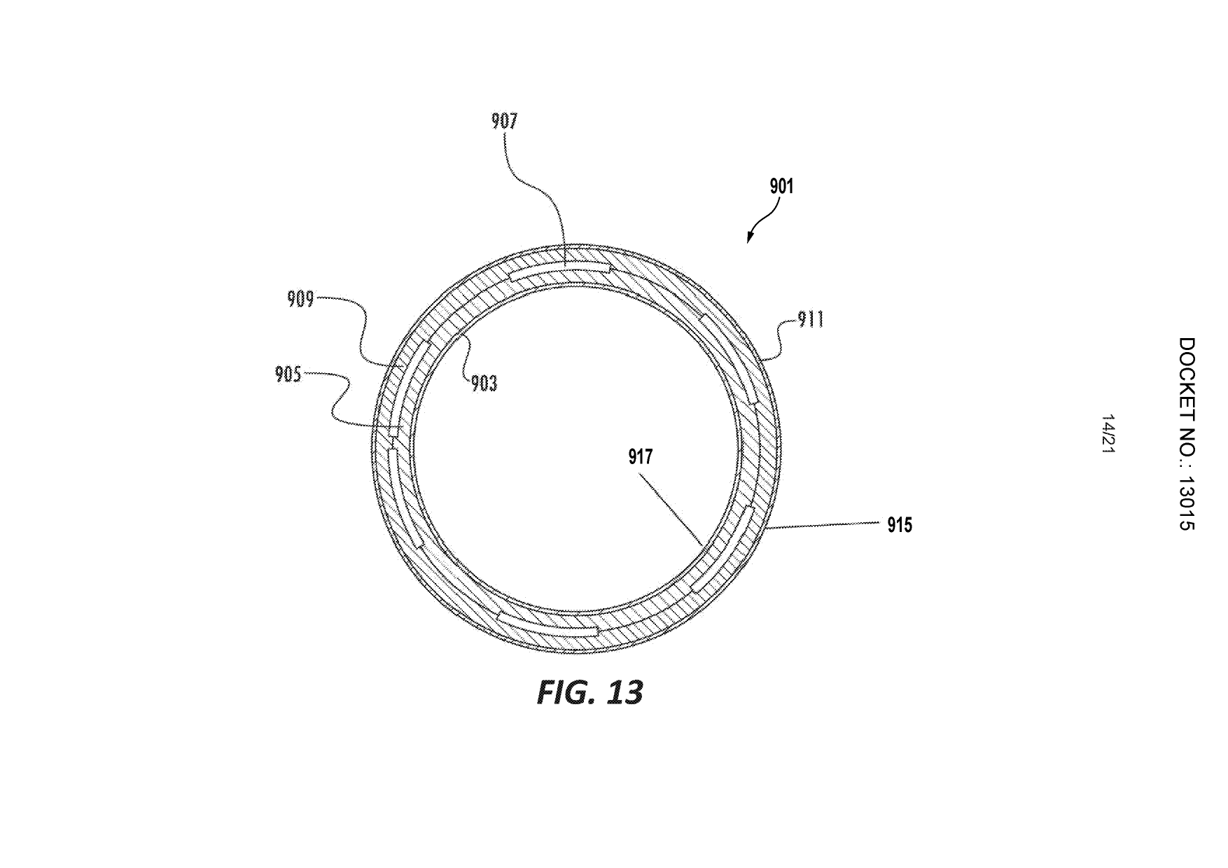

Some exemplary implementations are shown in FIGS. 13-17. An exemplary sheath disclosed herein has a proximal end and a distal end, an inner surface, and an outer surface. FIG. 13 shows a cross-section of such an exemplary sheath 901. The exemplary sheath 901 (FIG. 13) has an inner surface 917 and an outer surface 915.

In some implementations, an expandable sheath 901 shown in FIG. 13 further comprises an inner low-friction liner 903 having a first surface and an opposite second surface, where the first surface of the inner liner 903 defines the inner surface 917 of the sheath 901. It is understood that the inner liner can comprise one or more polymer layers. In some implementations, the inner liner can comprise two or more layers. In some implementations, the inner liner 903 can comprise from 1 to 8 layers, including an exemplary amount of 2, 3, 4, 5, 6, and 7 layers. It is understood that the inner liner can also comprise more than 8 layers, for example, and without limitation, it can comprise 9, 10, 15, 20, or more than 25 layers. It is understood that in some implementations, the layers of the polymers can be melted together during manufacturing processes.

In some implementations, if desired, an additional low friction polymer layer, such as PTFE, for example (not shown), can be disposed on the first surface of the inner liner 903. In such an exemplary implementation, the PTFE layer would define the inner surface of the sheath 901.

In some implementations, the exemplary sheath 901 further comprises an outer low-friction liner 911 having a first surface and an opposite second surface, wherein the second surface of the outer liner 911 defines the outer surface 915 of the sheath 901. It is also understood that similar to the inner liner 903, the outer liner 911 can comprise one or more polymer layers. Yet, in some implementations, the outer liner 911 can comprise two or more polymer layers. In some implementations, the outer liner can comprise from 1 to 8 layers, including an exemplary amount of 2, 3, 4, 5, 6, and 7 layers. It is understood that the outer liner 911 can also comprise more than 8 layers, for example, and without limitation, it can comprise 9, 10, 15, 20, or more than 25 layers. It is understood that in some implementations, the layers of the polymers can be melted together during manufacturing processes.

In some implementations, the outer liner 911 can further comprise any of the disclosed herein hydrophilic coatings.

The sheath 901 can further comprise a first polymeric layer 905 that surrounds radially outward of the inner liner 903, such that it overlies the second surface of the inner liner 903. In the implementations disclosed herein, the first polymeric layer 905 can comprise one or more sublayers. Yet, in some implementations, the first polymeric layer 905 can comprise two or more polymeric sublayers. For example, the first polymeric layer 905 can comprise from 1 to 8 sublayers, including an exemplary amount of 2, 3, 4, 5, 6, and 7 sublayers. It is understood that the first polymeric layer 905 can also comprise more than 8 sublayers, for example, and without limitation, it can comprise 9, 10, 15, 20, or more than 25 sublayers. It is understood that in some implementations, the sublayers of the first polymeric layer 905 can be melted together during manufacturing processes.

In some implementations, the sheath 901 can further comprise a braided layer 907 that is disposed radially outward of the first polymeric layer 905.

In some implementations, the sheath 901 can further comprise a second polymeric layer 909 that surrounds radially outward of the braided layer 907. In the implementations disclosed herein, the second polymeric layer 909 can comprise at least one sublayer or two or more polymeric sublayers. For example, the second polymeric layer can comprise from 1 to 8 sublayers, including an exemplary amount of 2, 3, 4, 5, 6, and 7 sublayers. It is understood that the second polymeric layer 909 can also comprise more than 8 sublayers, for example, and without limitation, it can comprise 9, 10, 15, 20, or more than 25 sublayers. It is understood that in some implementations, the sublayers of the second polymeric layer 909 can be melted together during manufacturing processes. As disclosed herein, the first surface of the outer liner 911 overlies the second polymeric layer 909.

FIG. 13 is shown as an exploded view for exemplary purposes. In practice, adjacent layers will be in contact with each other and, in some cases, laminated such that they meld with or extend through each other. In some implementations, the layers of this exemplary sheath 901 form a laminate structure.

For example, and without limitations, any of the disclosed herein braided layers can be used as the braided layer 907. In certain implementations, the braided layer 907 can comprise a plurality of helical multifiber filaments braided together. In such implementations, the first polymeric layer 905 and second polymeric layer 909 can be thermally bonded to each other through the open spaces of the braided layer 907 such that the braided layer 907 is encapsulated between these two polymeric layers. In some implementations, the first polymeric layer 905 and second polymeric layer 909 can also be thermally bonded to the adjacent inner liner 903 and outer liner 911. In such implementations, the braided layer 907 is encapsulated between all the layers of the sheath 901. In some implementations, and as disclosed herein, the inner liner 903 and outer liner 911 can comprise various polymeric materials. In some implementations, these polymeric materials can be porous. In such examples, the first polymeric layer 905 and second polymeric layer 909 can penetrate at least a portion of the pores present in the porous material of the inner liner 903 and/or outer liner 911 during the manufacturing process. As a result, the sheath 901 is more mechanically stable than any other sheaths known in the art.

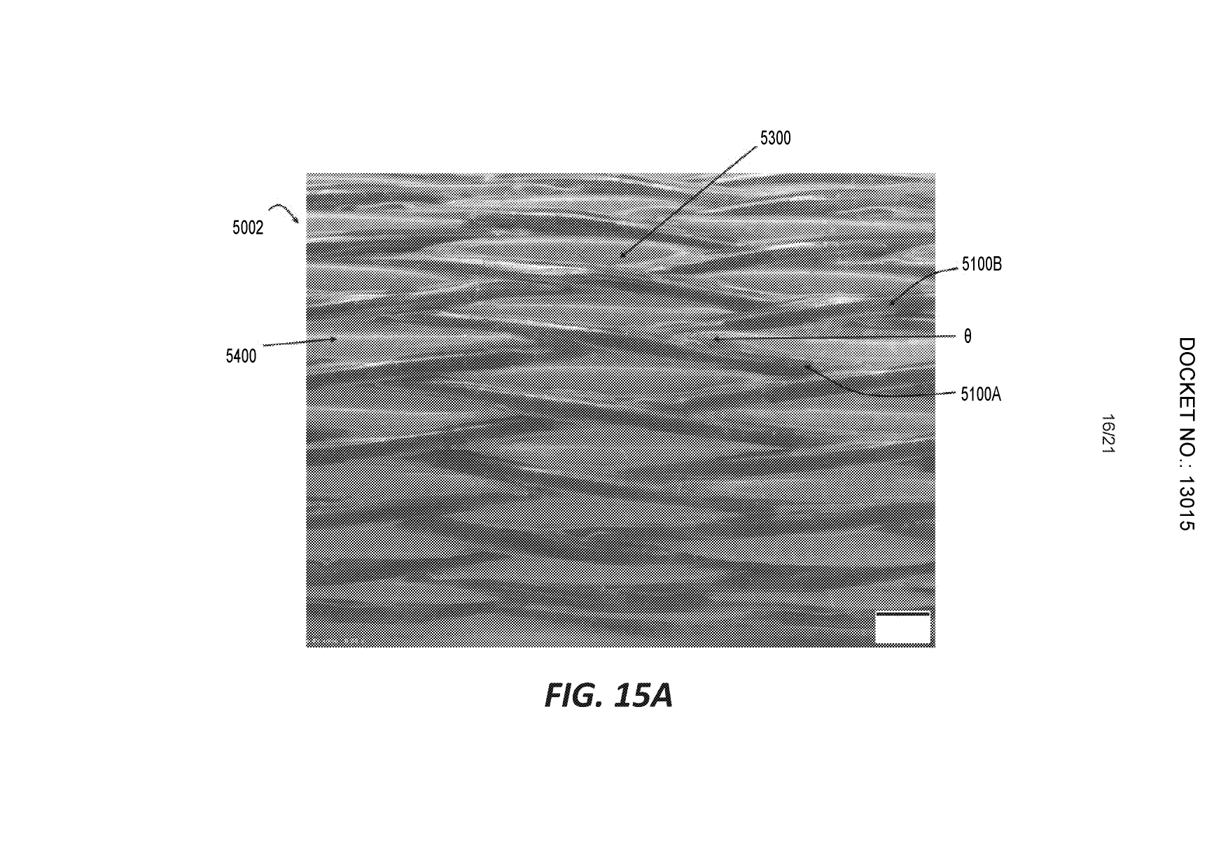

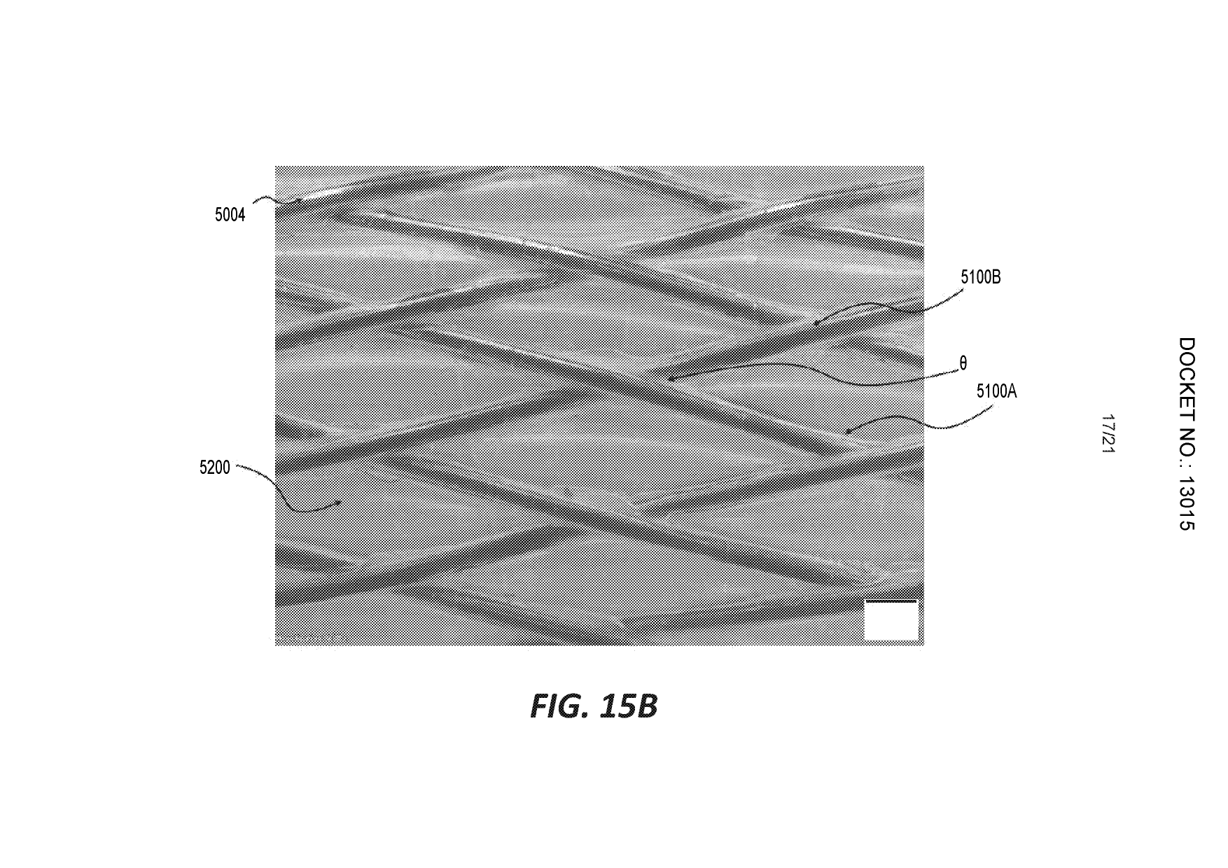

As disclosed in detail herein, when the first polymeric layer 905, second polymeric layer 909, inner liner 903, and outer liner 911 encapsulate the braided layer 907, they can connect (adhere or penetrate if the porous materials are present) to each other through the spaces between the filaments of the braided layer 907 as shown herein (for example as shown in FIGS. 15A-15B). In some implementations, the layers of the sheath 901 can also be bonded (adhered or penetrated into the pores if present) together at the proximal and/or distal ends of the sheath 901.

In some implementations, it is understood that the filaments of the braided layer 907 are not adhered to the polymeric layers of the sheath 901. This can allow the filaments, similarly to the implementations disclosed herein, to move angularly relative to each other and relative to the first polymeric layer 905 and the second polymeric layer 909, as well as relatively to all polymeric layers

of the laminate structure, allowing the diameter of the braided layer 907, and thereby the diameter of the sheath 901, to increase or decrease upon the passage of the medical device 12. Again, as disclosed herein, in this exemplary sheath, the angle θ between the filaments can change, and the length of the braided layer 907 can also change. For example, as the angle θ increases, the braided layer 907 can foreshorten, and as the angle θ decreases, the braided layer can lengthen to the extent permitted by the areas where the polymeric layers of the laminate structure are bonded. However, because the braided layer 907 is not adhered to the polymeric layers of the sheath, the change in length of the braided layer 907 that accompanies a change in the angle θ between the filaments does not result in a significant change in the length L of the sheath 901.

In some implementations, the laminate structure of this exemplary sheath 901 can facilitate resistance to kinking and ballooning.

In some implementations, the inner liner 903 and outer liner 911 can comprise any of the material disclosed herein. In certain implementations, the inner liner 903 comprises a first material. In some implementations, the outer liner 911 can comprise a fourth material. In certain implementations, the first and fourth materials can be the same or different.

In some implementations, the material (the first and the fourth) of a low friction inner liner 903 and outer liner 911 can be a material with a relatively low coefficient of friction yet a relatively high tensile strength. The inner liner 903 and/or outer liner 911 can have a coefficient of friction of less than about 0.3, less than about 0.2, less than about 0.1, less than about 0.09, less than about 0.08, less than about 0.07, less than about 0.06, less than about 0.05, less than about 0.04, less than about 0.03, less than about 0.02, or even less than about 0.01.

In some exemplary implementations, materials for the inner liner 903 and outer liner 911 (the first and the fourth) can comprise ultra-high molecular weight polyethylene (UHMWPE). In some implementations, the UHMWPE can be present as a fabric, a laminate, or a porous film or membrane. For example, the inner liner 903 and outer liner 911 can comprise or be formed of Dyneema® UHMWPE. In some implementations, the inner and outer liner can comprise, or be formed of, the Dyneema Purity® membrane, which has a tensile strength of about 20 MPa. In some exemplary and unlimiting implementations, the inner and outer liner can also be formed by coating. In such implementations, the UHMWPE can be provided as a polymeric solution, for example. Any known in the art coating methods can be utilized. For example, the coating methods can include dipping, doctor blade coating, spraying, and the like.

Other suitable materials for the inner liner 903 and outer liner 911 (in addition or instead of those disclosed above) can include polytetrafluoroethylene (PTFE), expanded polytetrafluoroethylene (ePTFE), ethylene tetrafluoroethylene (ETFE), nylon, polyethylene, polyether block amide (for example, Pebax), and/or combinations of any of the above. Again, it is understood that these materials can also be provided in any known in the art form.

In some implementations, the first polymeric layer 905 and the second polymeric layer 909 can also comprise any of the disclosed herein materials. In some implementations, the first polymeric layer 905 and the second polymeric layer 909 can be the same or different. In some implementations, the first polymeric layer 905 can comprise a second material, while the second polymeric layer 909 can comprise a third material. Again, as mentioned herein, the second material and the third materials can be the same or different. In certain implementations, the second material and/or the third material can comprise polyolefin or polyurethane. In some implementations, the polyolefin can comprise polyethylene, polypropylene, or a combination thereof. In some implementations, where the second material and/or the third material are polyolefin, such a polyolefin can comprise a bi-oriented polypropylene, cast polypropylene, a low-density polyethylene (LDPE), or a high density polyethylene (HDPE), or any combination thereof. In some implementations, where the second material and/or the third material are polyurethane, such implementations comprise a thermoplastic polyurethane.

It is understood that the second and/or the third materials can be provided in any form known in the art. In some implementations, they can be provided as a film or as a solution. In such implementations, if the materials are provided as a solution, the first and the second polymeric materials can be formed by coating, for example, dipping, spraying, doctor blade coating, and the like.

In some implementations, the tensile strength of the first polymeric layer 905 and/or the second polymeric layer 909 is substantially the same or different from the tensile strength of the inner liner 903 and/or outer liner 911. In some implementations, the tensile strength of the first polymeric layer 905 and/or the second polymeric layer 909 can be larger than the tensile strength of the inner liner 903 and/or outer liner 911. In some implementations, the tensile strength of the first polymeric layer 905 and/or the second polymeric layer 909 can be smaller than the tensile strength of the inner liner 903 and/or outer liner 911.

In some exemplary implementations, the porous structure of inner liner 903 and outer liner outer liner 911 can enable the first polymeric layer 905 and second polymeric layer 909 to flow into the pores during processing to mechanically bond the layers together. Such a laminate structure allows the sheath 901 to be more mechanically stable and durable. In such implementations, the inner liner 903 and the outer liner 911 can exhibit a mechanical strength higher than a mechanical strength of a reference sheath that does not comprise a substantially identical laminated structure. In some implementations, the disclosed herein expandable sheath 100 can exhibit an improved column strength when compared with a substantially identical reference sheath in the absence of a laminate structure.

In some implementations, the inner liner 903 and outer liner 911 can be relatively thin compared to the radial thickness of the adjacent first polymeric layer 905 and second polymeric layer 909, having the appearance of a liner or a membrane. For example, the inner liner 903 and/or outer liner 911 can have a radial thickness ranging from about 0.5 microns to about 40 microns, including

about 1 micron, about 2 microns, about 3 microns, about 4 microns, 5 microns, about 10 microns, about 15 microns, about 20 microns, about 25 microns, about 30 microns, about 35 microns, and about 40 microns.

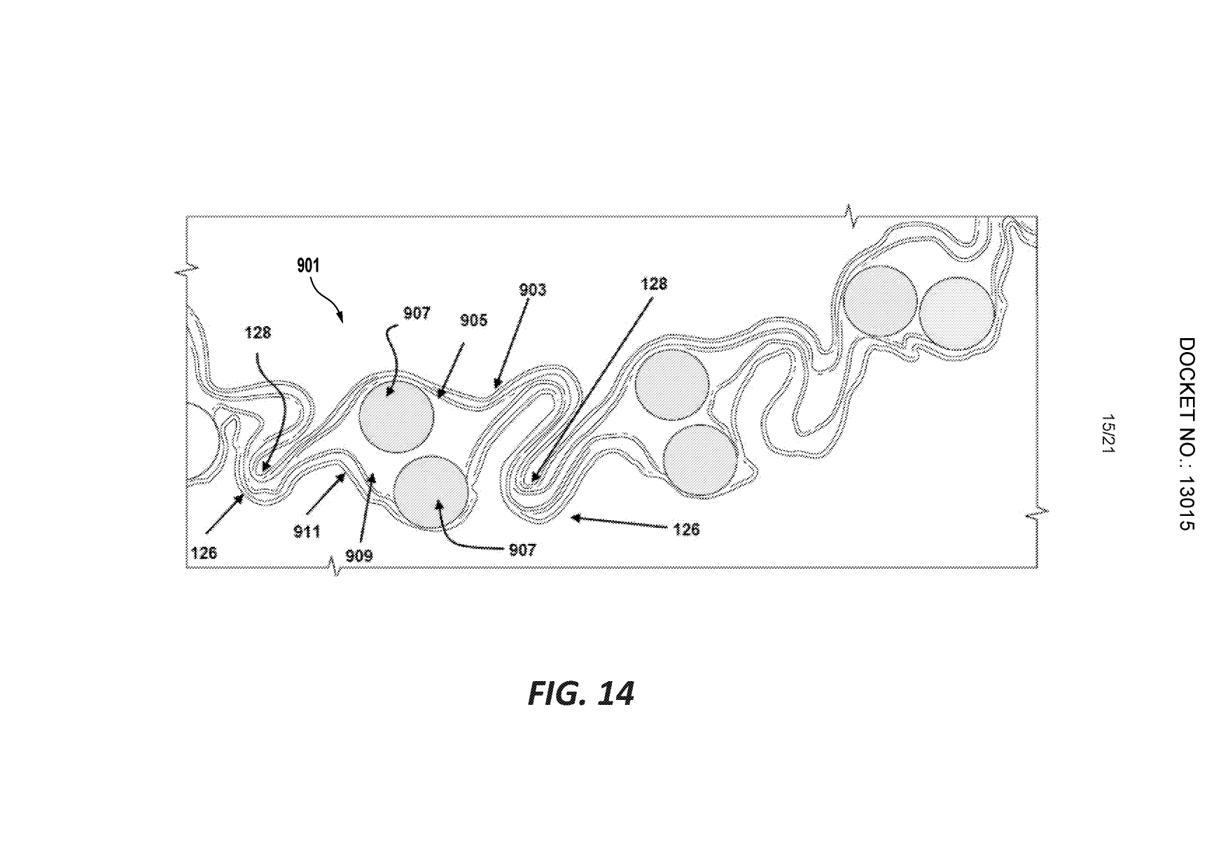

In some implementations and as illustrated in FIG. 14, the sheath 100 can comprise a plurality of pleats of creases that extend along at least a portion of the circumference of the sheath 100 and along its length. Similar pleats (folds, creases having ridges 126, and valleys 128) are shown in FIG. 8, for example. FIG. 14 shows a cross section of a portion of the sheath 100. It can be seen that the braid filaments of the braided layer 907 are encapsulated between inner liner 903 and outer liner 911 and the first polymeric layer 905 and the second polymeric layer 909, respectively. The pleats having ridges 126 and valleys 128 are spaced circumferentially along the sheath 100. It is understood that each of the plurality of pleats can comprise at least a portion of the inner liner 903, and/or at least a portion of the first polymeric layer 905, and/or at least a portion of the braided layer 907, and/or at least a portion of the second polymeric layer 909, and/or at least a portion of the outer liner 911. These pleats are configured to flatten out when the sheath 901 is in an expended state and form back after collapsing back to the unexpended state. It is understood that these pleats can have a random pattern around the circumference of the sheath 901 or along a length of the sheath 901. It is also understood that these pleats can have a random longitudinal pattern that is not the same at different portions of the sheath’s 901 length. In some implementations, the plurality of pleats can extend along just a central portion, along just a proximal portion, or close to a distal portion, or along of any combination thereof.

The plurality of pleats, as shown in FIG. 14, in some implementations, can extend longitudinally along just a portion of the sheath or along the entire length of the sheath 901. When the sheath 901 is in a collapsed (unexpanded state, the circumferentially spaced pleats can form a plurality of ridges 126 circumferentially spaced apart from each other by valleys 128. It is understood that these circumferentially spaced pleats can be extended longitudinally in an orderly or random fashion.

Yet, in some implementations, the pleats can be structured circumferentially or longitudinally in any desired pattern. It is also understood that these pleats along the length of the sheath 901 are formed as the various polymer layers encapsulate the braid during the manufacturing proceedings and are flattening out or shortened as a result of braid expansion.

In other words, in some implementations, the plurality of pleats can be uniformly distributed along at least a portion of a length of the sheath 901 and at least a portion of a circumference of the sheath 901; while in some implementations, the plurality of pleats can be randomly distributed along at least a portion of a length of the sheath 901 and at least a portion of a circumference of the sheath 901.

In some implementations, as the medical device 12 is passed through the sheath 901, the ridges 126 and valleys 128 of the pleats can at least partially level out to allow a sheath wall to

radially expand and allow the medical device 12 to pass through without damaging the sheath 901 or vascular system of the patient. The photograph of the locally collapsed and locally expanded sheath 901 is shown in FIGS. 15A-15B, respectively.

It can be seen in FIG. 15A that in the collapsed state, the sheath 5002 comprises a braided layer having the filaments 5100A and 5100B (arranged similar to the filaments shown in FIG. 10B) having an angle θ. The plurality of pleats form ridges 5400 and valleys 5300 around the filaments 5100A and 5100B of the braided layer. When the sheath 5002 is in the expanded state 5004, as shown in FIG. 15B, the plurality of the pleats straightens out as shown in 5200, while the angle θ between the filaments 5100A and 5100B increases. It is understood that angle θ can have any value between about 5° to about 70°, including exemplary values of about 10°, about 15°, about 20°, about 25°, about 30°, about 35°, about 40°, about 45°, about 50°, about 55°, about 60°, and about 65° depending on whether the sheath 5002 in the collapsed, partially collapsed, partially expanded, or expanded state.

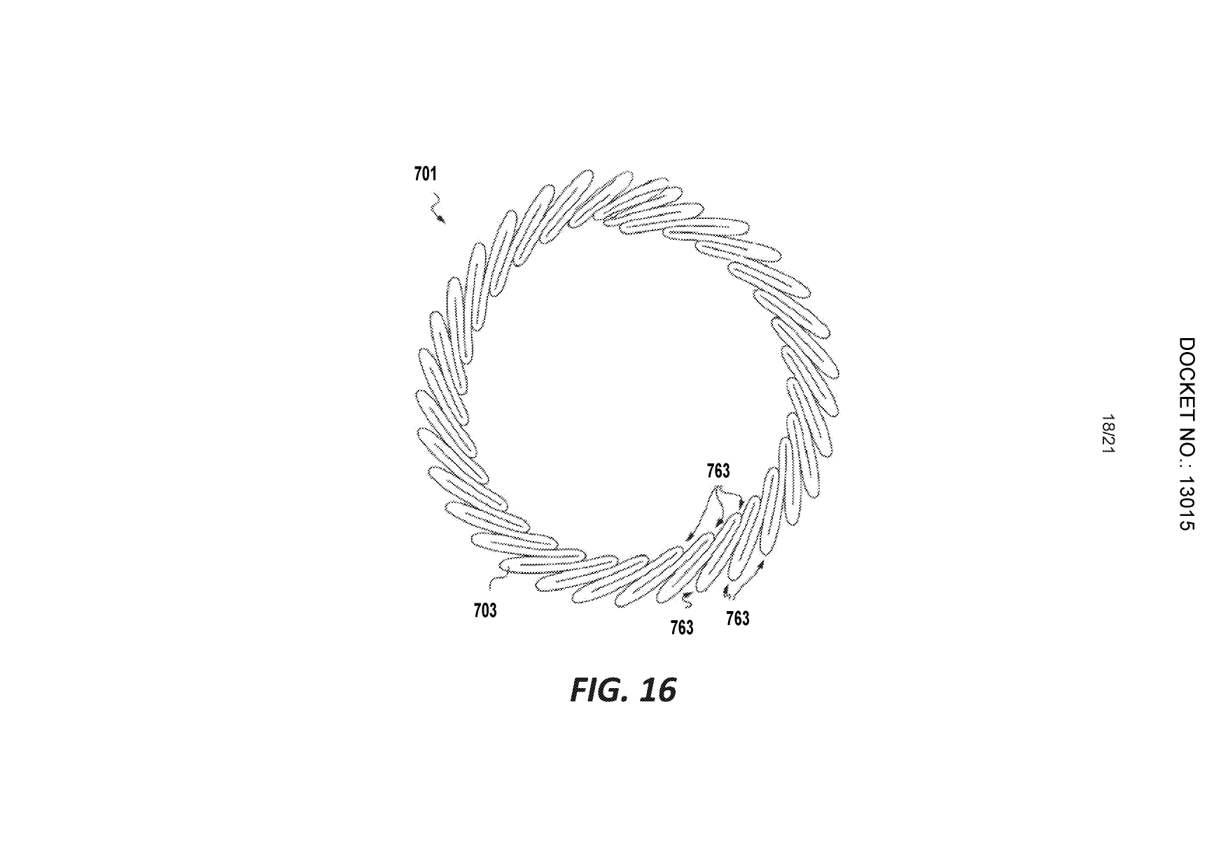

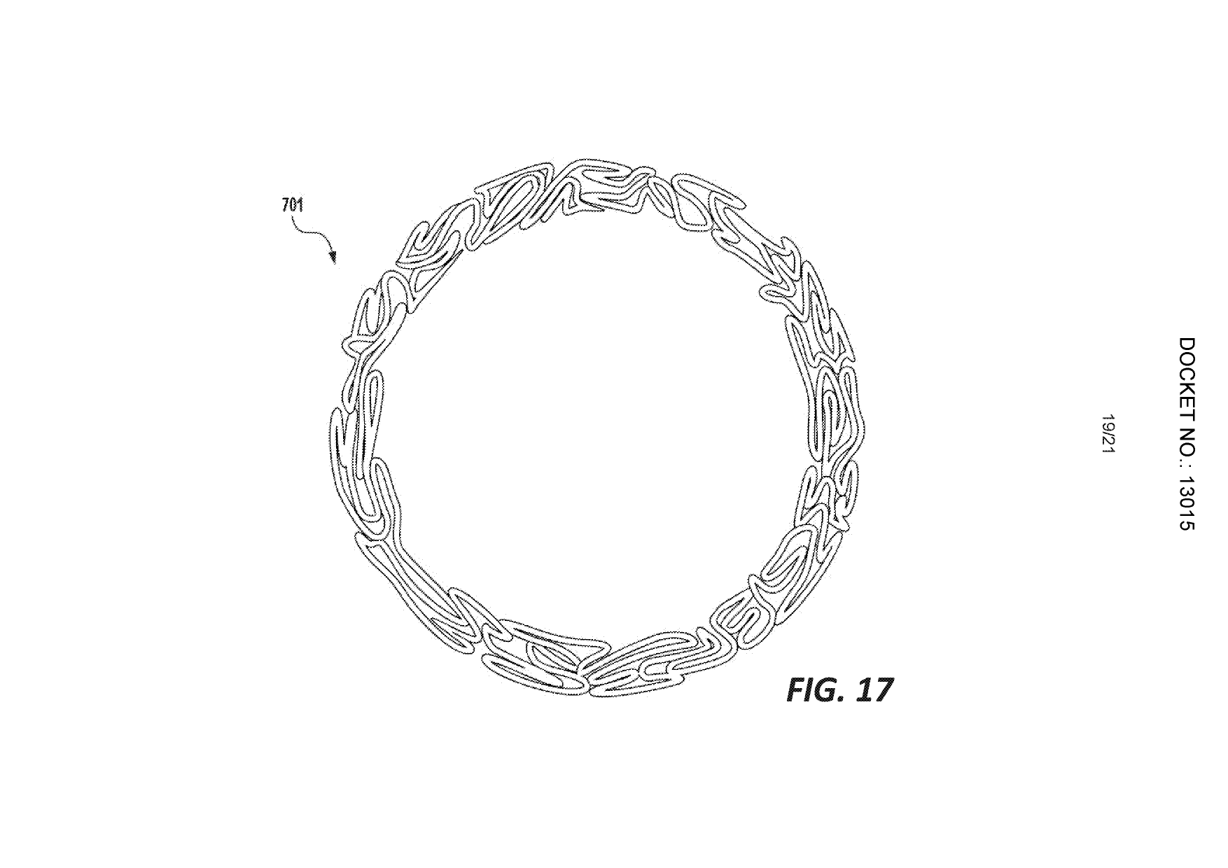

FIGS. 16 and 17 show a distal end or a tip of an example sheath 701. On the distal end of the sheath 701, the pleats can have a more organized structure of the pleats. It is understood that the sheath’s tip may not comprise a braid, and therefore, the pleats are not dependent on the braid structure and the state. As shown, the tip (or distal portion of the sheath 701) can comprise a plurality of longitudinally extending pleats (fold). As shown in FIG. 16, the collapsed (unexpanded) wall 703 of a sheath 701 can include a plurality of pleats 763. It is understood that each of the plurality of pleats can comprise at least a portion of the inner liner 903, at least a portion of the first polymeric layer 905, at least a portion of the second polymeric layer 909, and at least a portion of the outer liner 911.

The folds can be arranged in an orderly fashion, as shown in FIG. 16, but are not necessarily so. In some implementations, manufacturing processes lead to more randomly arranged longitudinally-extending folds, valleys, and ridges, as shown in FIG. 17.

As a medical device (for example, medical device 12) passes through the inner lumen of the tip, it applies an outward radial force on the sheath wall 703. This causes the plurality of longitudinally-extending pleats 763 to partially or fully unfold (or at least partially smooth out) (that is, a single longitudinally-extending pleat may partially or fully unfold, and several of the longitudinally-extending pleats may partially or fully unfold, or all of the longitudinally-extending pleats may partially or fully unfold).

In some implementations, an additional lubricious liner can be used. In such implementations, this liner can be applied to the first surface of the sheath and becomes the most inner surface of the sheath 701. In such implementations, this additional lubricious liner can comprise a low coefficient of friction materials that can facilitate the passage of the medical device 12 through the inner lumen.

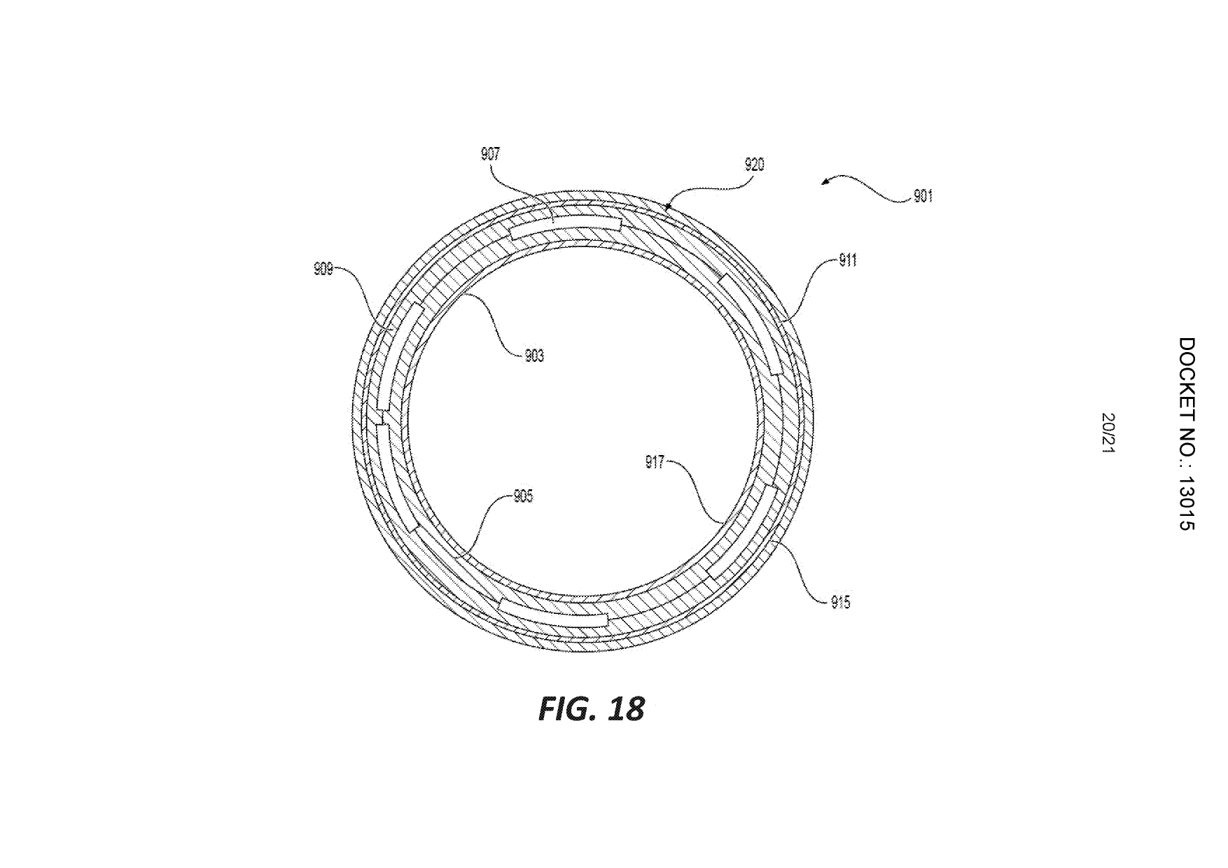

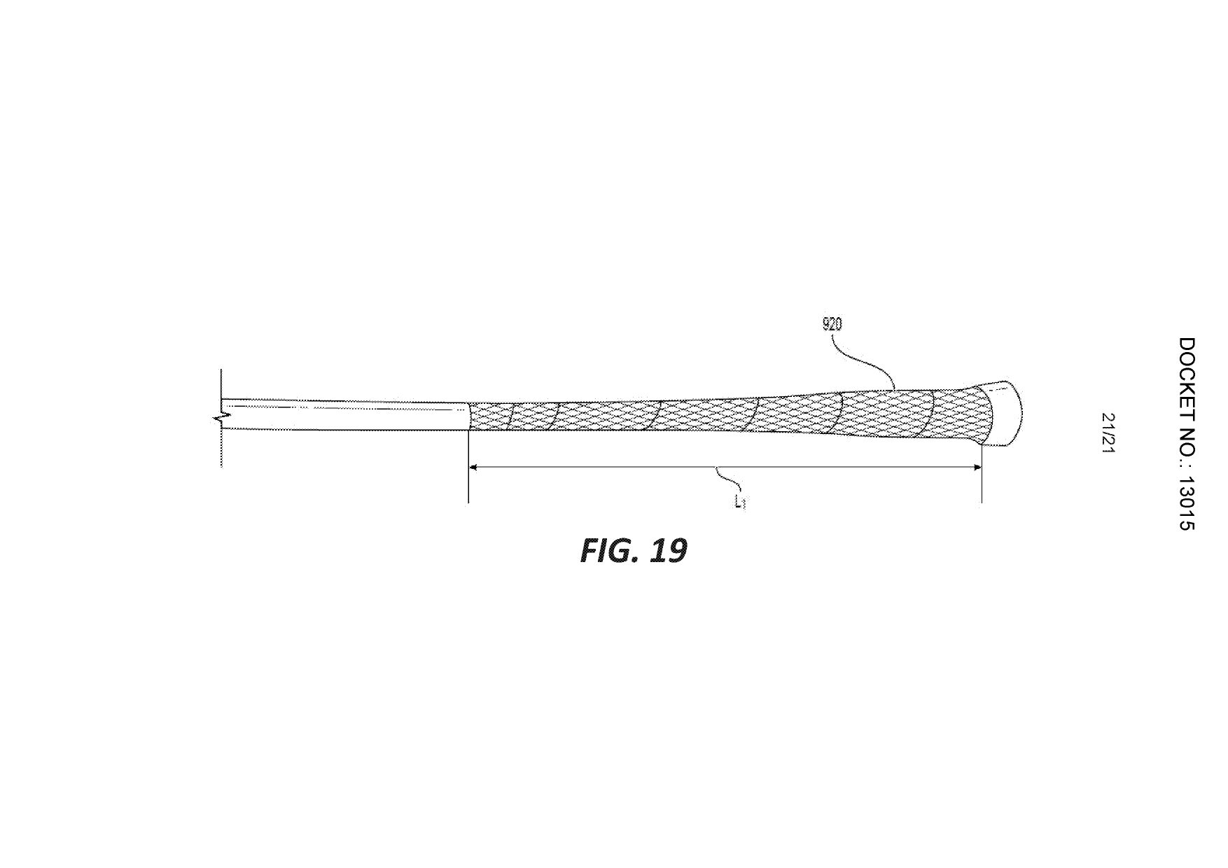

An additional implementation of the disclosed sheath is shown in FIG. 18. In this implementation, a third polymeric layer 920 comprising one or more layers can be disposed on at least a portion of the proximal end of the sheath 901. A photograph of such an implementation is shown in FIG. 19, where L1 shows a length of the third polymeric layer in the proximal portion. In such an implementation, a fifth material is wrapped radially outward of at least a portion of the outer liner 911 to form the third polymeric layer. In certain implementations, the fifth material is wrapped at least 2 times around the portion of the outer liner 911. Yet, in some implementations, the fifth material can be wrapped multiple times, forming from about 2 to about 10 layers.

In some implementations, the fifth material can be any material that is suitable for the desired application. Yet, in some implementations, the fifth material can comprise ultra-high molecular weight polyethylene (UHMWPE). In some implementations, the UHMWPE can be present as a fabric, a laminate, or a porous film or membrane. For example, the fifth material forming the third polymeric layer can comprise or be formed of Dyneema® UHMWPE. In some implementations, the third polymeric layer can comprise, or be formed of, the Dyneema Purity® membrane, which has a tensile strength of about 20 MPa.

In some exemplary and unlimiting implementations, the third polymeric layer can also be formed by coating. In such implementations, the UHMWPE can be provided as a polymeric solution, for example. Any known in the art coating methods can be utilized. For example, the coating methods can include dipping, doctor blade coating, spraying, and the like.

It is understood that in implementations where the third polymeric layer is present, such a layer is formed after the laminate structure described herein and comprising the inner liner 903, the first polymeric layer 905, the braided layer 907, the second polymeric layer 909, and the outer liner 911 is formed.

In some implementations, after the third polymeric layer is formed either by wrapping the fifth material or by coating, a structure is heated to a temperature from about 120 °C to about 150 °C, including exemplary values of about 125 °C, about 130 °C, about 135 °C, about 140 °C, and about 145 °C. It is understood that at such temperatures, the third material is not fully melted and therefore is not expected to penetrate all the layers beneath it, and more specifically, it is not expected to be bound to the braided layer. In some implementations, at such temperatures, the third polymeric layer is at least partially bonded to the outer liner 911.

In certain implementations, prior to the exposure of the third polymeric layer to the described above temperature, a sacrificial heat tubing is first disposed on the third polymeric layer, and then the sheath 901 is exposed to elevated temperatures. Yet, in some implementations, the heating of the third polymeric layer to form the sheath 901 described herein can be performed without the presence of the sacrificial sheath tubing.

In some implementations, where the third polymeric layer is laminated to at least a portion of the outer liner 911, an outer surface of the third polymeric layer is smoother than an outer surface of the outer liner 911 anywhere on the sheath 901. In some implementations, an outer surface of the third polymeric layer exhibits less roughness than an outer surface of the outer liner 911 anywhere on the sheath 901.

In yet some implementations, the third polymeric layer exhibits higher porosity than the outer liner 911.

In some implementations, the at least portion of the proximal end of the sheath 901 that is covered by the third polymeric layer is up to about 15 cm, including exemplary values of about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, and about 14 cm.

In some implementations, at least a portion of the sheath 901 with the third polymeric layer is inserted into a patient’s body. In such implementations, the third polymeric layer can form a substantial seal with the patient’s natural anatomy to prevent unnecessary blood loss.

In view of the many possible implementations to which the principles of the disclosed disclosure can be applied, it should be recognized that the illustrated implementations are only preferred examples of the disclosure and should not be taken as limiting the scope of the disclosure. Rather, the scope of the disclosure is defined by the following claims. We, therefore, claim as our disclosure all that comes within the scope and spirit of these claims.

### Exemplary Aspects:

In view of the described processes and compositions, below are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An introducer assembly including: an elongated introducer including a central lumen that extends between a proximal opening at proximal end of the introducer and a distal opening at a distal end of the introducer; and an elongated core member received within the central lumen of the introducer and movable between a first position and a second position, the core member including: an elongated body portion; and an expansion element extending radially from an outer surface of the body portion; wherein at least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the central lumen of the introducer by the expansion element as it moved between the first and second positions.

Example 2: The introducer assembly according to any example provided herein, particularly example 1, wherein, when the core member is in the first position, at least a portion of the expansion element and/or the body portion extends through the distal opening, wherein, when the core member is in the second position, the expansion element is positioned within the central lumen of the introducer such that contact between the expansion element and the central lumen provides an outwardly directed radial force on the central lumen moving the introducer from the unexpanded configuration to the expanded configuration.

Example 3: The introducer assembly according to any example provided herein, particularly examples 1-2, wherein the core member includes a central lumen extending between a proximal end and a distal end of the core member.

Example 4: The introducer assembly according to any example provided herein, particularly examples 1-3, wherein the expansion element includes distal tapered surface adjacent the distal end of the of the core member.

Example 5: The introducer assembly according to any example provided herein, particularly examples 1-4, wherein the expansion element includes a proximal tapered surface extending between the elongated body portion of the core member and an increased diameter portion of the expansion element.

Example 6: The introducer assembly according to any example provided herein, particularly examples 1-5, wherein the expansion element has a barrel-shaped longitudinal cross section.

Example 7: The introducer assembly according to any example provided herein, particularly examples 1-6, wherein the introducer includes a plurality of slits extending longitudinally along the introducer and extending radially from an inner surface of the central lumen, where separation/widening of at least one of the plurality of slits facilitates expansion of the introducer from the unexpanded configuration to the expanded configuration, wherein the plurality of slits are evenly spaced around a circumference of the central lumen.

Example 8: The introducer assembly according to any example provided herein, particularly examples 1-7, further including: an introducer locking mechanism for fixing an axial/longitudinal location of the introducer with respect to the core member, and a sheath locking mechanism for fixing an axial/longitudinal location of the introducer with respect to an expandable sheath.

Example 9: A method of expanding a sheath comprising: positioning an elongated core member within a central lumen of an elongated introducer in a first position, where the core member includes an elongated body portion and an expansion element extending radially from an outer surface of the body portion; advancing the core member and the introducer into a central lumen of an expandable sheath; expanding the introducer by moving the core member into a second position such that the introducer moves from an unexpanded configuration in which the introducer has a first outer diameter to an expanded configuration in which the introducer has a second, larger, outer diameter due to an outwardly directed radial force exerted on the central lumen of introducer by the expansion element as it moved from the first position to the second position; and expanding the expandable sheath by moving the core member within the central lumen of the introducer such that contact between the expansion element and the central lumen provides an outwardly directed radial force on the central lumen moving the introducer from the unexpanded configuration to the expanded configuration resulting in a corresponding radial expansion along a length of the sheath.

Example 10. The method according to any example provided herein, particularly example 9, wherein positioning the core member at the second position within the central lumen of the elongated introducer includes advancing the core member within the introducer such that at least a portion of the expansion element and/or the body portion extends through a distal opening of the introducer.

Example 11. The method according to any of the examples provided herein, particularly examples 9-10, wherein expanding the expandable sheath further includes moving the core member and introducer together within the central lumen of the sheath such that contact between the expanded introducer and the sheath results in a corresponding radial expansion of the sheath.

Example 12. The method according to any of the examples provided herein, particularly examples 9-11, further including coupling the core member to the introducer in the first position fixing an axial and/or rotational position of the core member with respect to the introducer and preventing corresponding movement therebetween.

Example 13. The method according to any of the examples provided herein, particularly examples 9-12, wherein, before expanding the expandable sheath by moving the core member to the second position, the introducer is coupled to the sheath fixing an axial and/or rotational position of the introducer with respect to the sheath and preventing corresponding movement therebetween.

Example 14. The method according to any of the examples provided herein, particularly examples 9-13, further including: advancing the combined core member, introducer and sheath into a patient's blood vessel with the core member in the first position with respect to the introducer; expanding a length of the blood vessel by expanding a corresponding length of the expandable sheath; withdrawing the introducer and core member from the blood vessel; and advancing a medical device through the central lumen of the sheath and beyond a distal opening of the sheath to a treatment site, wherein the medical device is a prosthetic heart valve mounted in a radially crimped state on a delivery apparatus, and the method further comprises implanting the prosthetic heart valve at the treatment site within the patient.

Example 15. An introducer system including: an elongated introducer including: a central lumen that extends between a proximal opening at proximal end of the introducer and a distal opening at a distal end of the introducer; and an inner shoulder extending radially inward from the central lumen; and an elongated core member received within the central lumen of the introducer and movable between a first position and a second position, the core member including an elongated body portion; wherein at least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the inner shoulder of the introducer by the core member as it moved between the first and second positions.

Example 16. The introducer system according to any of the examples provided herein, particularly example 15, wherein, when the core member is in the first position, the distal end of the core member is positioned proximal of the inner shoulder, wherein, when the core member is in the second position, the core member is positioned adjacent the inner shoulder such that contact between the core member and the inner shoulder provides an outwardly directed radial force on the introducer moving the introducer from the unexpanded configuration to the expanded configuration.

Example 17. The introducer system according to any of the examples provided herein, particularly examples 15-16, wherein the core member includes a central lumen extending between a proximal end and distal end of the core member, wherein the core member has a constant outer diameter along a length of the core member, wherein the core member includes a distal tapered surface that extends from the distal end of the core member to facilitate advancement of the core member along the inner shoulder.

Example 18. The introducer system according to any of the examples provided herein, particularly examples 15-17, wherein the inner shoulder extends longitudinally from a distal end of the introducer and defines a reduced diameter portion of the central lumen having a diameter less than a diameter of a main diameter portion of the central lumen when the introducer is in the unexpanded configuration.

Example 19. The introducer system according to any of the examples provided herein, particularly example 18, wherein the inner shoulder has a proximal tapered surface extending between the main diameter portion and the reduced diameter portion facilitating sliding movement between the core member and the inner shoulder as the core member is moved distally during introducer expansion.

Example 20. The introducer system according to any of the examples provided herein, particularly examples 15-19, wherein the introducer includes a plurality of slits extending longitudinally along the inner shoulder and extending radially from an inner surface of the central lumen, where separation/widening of at least one of the plurality of slits facilitates expansion of the introducer from the unexpanded configuration to the expanded configuration, wherein the plurality of slits are evenly spaced around a circumference of the central lumen.

Example 21. The introducer system according to any of the examples provided herein, particularly examples 15-20, further including: an introducer locking mechanism for fixing an axial/longitudinal location of the introducer with respect to the core member, and a sheath locking mechanism for fixing the axial/longitudinal location of the introducer with respect to an expandable sheath.

Example 22. A method of expanding a sheath comprising: positioning an elongated core member within a central lumen of an elongated introducer in a first position, the introducer including: a central lumen that extends between a proximal opening at proximal end of the introducer, a distal opening at a distal end of the introducer; and an inner shoulder extending radially inward from the central lumen; coupling the core member to the introducer in the first position fixing the position of the core member with respect to the introducer; advancing the core member and the introducer into a central lumen of an expandable sheath; coupling the introducer and/or core member to the expandable sheath fixing the position of the unexpanded introducer assembly with respect to the sheath; uncoupling the core member from the introducer such that the core member can freely move with respect to the introducer; expanding the introducer by moving the core member from the first position, where a distal end of the core member is positioned proximal of the inner shoulder, into a second position where the core member is advanced with the central lumen and in contact with the inner shoulder, such that the introducer moves from an unexpanded configuration in which the introducer has a first outer diameter to an expanded configuration in which the introducer has a second, larger, outer diameter due to an outwardly directed radial force exerted on the inner shoulder by the core member; uncoupling the introducer and/or core member from the expandable sheath; and expanding the expandable sheath by moving the core member and the introducer in the expanded configuration within the central lumen of the sheath such that contact between the expanded introducer and the sheath results in a corresponding radial expansion of the sheath.

Example 23. The introducer system according to any of the examples provided herein, particularly example 22, further including: advancing the combined core member, introducer and sheath into a patient's blood vessel with the core member in the first position with respect to the introducer; expanding a length of the blood vessel by expanding a corresponding length of the expandable sheath; withdrawing the introducer and core member from the blood vessel; and advancing a medical device through the central lumen of the sheath and beyond a distal opening of the sheath to a treatment site, wherein the medical device is a prosthetic heart valve mounted in a radially crimped state on a delivery apparatus, and the method further comprises implanting the prosthetic heart valve at the treatment site within the patient.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

We claim:

1. An introducer assembly including:
an elongated introducer including a central lumen that extends between a proximal opening at proximal end of the introducer and a distal opening at a distal end of the introducer; and
an elongated core member received within the central lumen of the introducer and movable between a first position and a second position, the core member including:
an elongated body portion; and
an expansion element extending radially from an outer surface of the body portion;
wherein at least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the central lumen of the introducer by the expansion element as it moved between the first and second positions.

2. The introducer assembly of claim 1, wherein, when the core member is in the first position, at least a portion of the expansion element and/or the body portion extends through the distal opening,
wherein, when the core member is in the second position, the expansion element is positioned within the central lumen of the introducer such that contact between the expansion element and the central lumen provides an outwardly directed radial force on the central lumen moving the introducer from the unexpanded configuration to the expanded configuration.

3. The introducer assembly of claim 1, wherein the core member includes a central lumen extending between a proximal end and a distal end of the core member.

4. The introducer assembly of claim 1, wherein the expansion element includes distal tapered surface adjacent the distal end of the core member.

5. The introducer assembly of claim 1, wherein the expansion element includes a proximal tapered surface extending between the elongated body portion of the core member and an increased diameter portion of the expansion element.

6. The introducer assembly of claim 1, wherein the expansion element has a barrel-shaped longitudinal cross section.

7. The introducer assembly of claim 1, wherein the introducer includes a plurality of slits extending longitudinally along the introducer and extending radially from an inner surface of the central lumen, where separation/widening of at least one of the plurality of slits facilitates expansion of the introducer from the unexpanded configuration to the expanded configuration,
wherein the plurality of slits are evenly spaced around a circumference of the central lumen.

8. The introducer assembly of claim 1, further including:
an introducer locking mechanism for fixing an axial/longitudinal location of the introducer with respect to the core member, and
a sheath locking mechanism for fixing an axial/longitudinal location of the introducer with respect to an expandable sheath.

9. A method of expanding a sheath comprising:
positioning an elongated core member within a central lumen of an elongated introducer in a first position, where the core member includes an elongated body portion and an expansion element extending radially from an outer surface of the body portion;
advancing the core member and the introducer into a central lumen of an expandable sheath;
expanding the introducer by moving the core member into a second position such that the introducer moves from an unexpanded configuration in which the introducer has a first outer diameter to an expanded configuration in which the introducer has a second, larger, outer diameter due to an outwardly directed radial force exerted on the central lumen of introducer by the expansion element as it moved from the first position to the second position; and
expanding the expandable sheath by moving the core member within the central lumen of the introducer such that contact between the expansion element and the central lumen provides an outwardly directed radial force on the central lumen moving the introducer from the unexpanded configuration to the expanded configuration resulting in a corresponding radial expansion along a length of the sheath.

10. The method of claim 9, wherein positioning the core member at the second position within the central lumen of the elongated introducer includes advancing the core member within the introducer such that at least a portion of the expansion element and/or the body portion extends through a distal opening of the introducer.

11. The method of claim 9, wherein expanding the expandable sheath further includes moving the core member and introducer together within the central lumen of the sheath such that contact between the expanded introducer and the sheath results in a corresponding radial expansion of the sheath.

12. The method of claim 9, further including coupling the core member to the introducer in the first position fixing an axial and/or rotational position of the core member with respect to the introducer and preventing corresponding movement therebetween.

13. The method of claim 9, wherein, before expanding the expandable sheath by moving the core member to the second position, the introducer is coupled to the sheath fixing an axial and/or rotational position of the introducer with respect to the sheath and preventing corresponding movement therebetween.

14. An introducer system including:
an elongated introducer including:
a central lumen that extends between a proximal opening at proximal end of the introducer and a distal opening at a distal end of the introducer; and
an inner shoulder extending radially inward from the central lumen; and
an elongated core member received within the central lumen of the introducer and movable between a first position and a second position, the core member including an elongated body portion;
wherein at least a portion of the introducer is configured to expand from an unexpanded configuration in which the introducer has a first outer diameter (D1) to an expanded configuration in which the introducer has a second, larger, outer diameter (D2) due to an outwardly directed radial force exerted on the inner shoulder of the introducer by the core member as it moved between the first and second positions.

15. The introducer system of claim 14, wherein, when the core member is in the first position, the distal end of the core member is positioned proximal of the inner shoulder,
wherein, when the core member is in the second position, the core member is positioned adjacent the inner shoulder such that contact between the core member and the inner shoulder provides an outwardly directed radial force on the introducer moving the introducer from the unexpanded configuration to the expanded configuration.

16. The introducer system of claim 14, wherein the core member includes a central lumen extending between a proximal end and distal end of the core member,
wherein the core member has a constant outer diameter along a length of the core member,
wherein the core member includes a distal tapered surface that extends from the distal end of the core member to facilitate advancement of the core member along the inner shoulder.

17. The introducer system of claim 14, wherein the inner shoulder extends longitudinally from a distal end of the introducer and defines a reduced diameter portion of the central lumen having a diameter less than a diameter of a main diameter portion of the central lumen when the introducer is in the unexpanded configuration,
wherein the inner shoulder has a proximal tapered surface extending between the main diameter portion and the reduced diameter portion facilitating sliding movement between the core member and the inner shoulder as the core member is moved distally during introducer expansion.

18. The introducer system of claim 14, wherein the introducer includes a plurality of slits extending longitudinally along the inner shoulder and extending radially from an inner surface of the central lumen, where separation/widening of at least one of the plurality of slits facilitates expansion of the introducer from the unexpanded configuration to the expanded configuration,
wherein the plurality of slits are evenly spaced around a circumference of the central lumen.

19. The introducer system of claim 14, further including:
an introducer locking mechanism for fixing an axial/longitudinal location of the introducer with respect to the core member, and
a sheath locking mechanism for fixing the axial/longitudinal location of the introducer with respect to an expandable sheath.

20. A method of expanding a sheath comprising:
positioning an elongated core member within a central lumen of an elongated introducer in a first position, the introducer including:
a central lumen that extends between a proximal opening at proximal end of the introducer, a distal opening at a distal end of the introducer; and
an inner shoulder extending radially inward from the central lumen;
coupling the core member to the introducer in the first position fixing the position of the core member with respect to the introducer;
advancing the core member and the introducer into a central lumen of an expandable sheath;
coupling the introducer and/or core member to the expandable sheath fixing the position of the unexpanded introducer assembly with respect to the sheath;
uncoupling the core member from the introducer such that the core member can freely move with respect to the introducer;
expanding the introducer by moving the core member from the first position, where a distal end of the core member is positioned proximal of the inner shoulder, into a second position where the core member is advanced with the central lumen and in contact with the inner shoulder, such that the introducer moves from an unexpanded configuration in which the introducer has a first outer diameter to an expanded configuration in which the introducer has a second, larger, outer diameter due to an outwardly directed radial force exerted on the inner shoulder by the core member;
uncoupling the introducer and/or core member from the expandable sheath; and
expanding the expandable sheath by moving the core member and the introducer in the expanded configuration within the central lumen of the sheath such that contact between the expanded introducer and the sheath results in a corresponding radial expansion of the sheath.
